(19) United States
(12) Patent Application Publication
(10) Pub. No.: US 2022/0017487 A1
(43) Pub. Date: Jan. 20, 2022

(54) DEUTERATED COMPOUNDS AND USES THEREOF

(71) Applicant: X4 Pharmaceuticals, Inc., Cambridge, MA (US)

(72) Inventors: Elyse Marie Josee Bourque, L'Etang-du-Nord (CA); Renato Skerlj, West Newton, MA (US)

(21) Appl. No.: 17/377,328

(22) Filed: Jul. 15, 2021

Related U.S. Application Data
(60) Provisional application No. 63/052,716, filed on Jul. 16, 2020.

(51) Int. Cl. C07D 401/12; C07B 59/00; A61P 35/00

(52) U.S. Cl. CPC C07D 401/12; C07B 2200/05; A61P 35/00; C07B 59/002

(57) ABSTRACT

The present invention provides deuterium-enriched CXCR4 inhibitors, pharmaceutical compositions, and methods of using such deuterium-enriched CXCR4 inhibitors and pharmaceutical compositions to treat, prevent, or ameliorate a disease, disorder, or condition such as cancer or WHIM syndrome.

### CROSS-REFERENCE TO RELATED APPLICATIONS

The present application claims priority to U.S. provisional patent application Ser. No. 63/052,716, filed Jul. 16, 2020, the entirety of which is incorporated herein by reference.

### TECHNICAL FIELD OF THE INVENTION

The present invention provides deuterium-enriched CXCR4 inhibitors, pharmaceutical compositions, and methods of using such deuterium-enriched CXCR4 inhibitors and pharmaceutical compositions to treat, prevent, or ameliorate a disease, disorder, or condition such as a cancer or a primary immunodeficiency.

### BACKGROUND OF THE INVENTION

C-X-C chemokine receptor type 4 (CXCR4), also known as fusin or cluster of differentiation 184 (CD184), is a seven transmembrane G-protein coupled receptor (GPCR) belonging to Class I GPCR or rhodopsin-like GPCR family. Under normal physiological conditions, CXCR4 carries out multiple roles and is principally expressed in the hematopoietic and immune systems. CXCR4 was initially discovered as one of the co-receptors involved in human immunodeficiency virus (HIV) cell entry. Subsequent studies showed that it is expressed in many tissues, including brain, thymus, lymphatic tissues, spleen, stomach, and small intestine, and also specific cell types such as hematopoietic stem cells (HSC), mature lymphocytes, and fibroblasts. CXCL12, previously designated SDF-1α, is the only known ligand for CXCR4. CXCR4 mediates migration of stem cells during embryonic development as well as in response to injury and inflammation. Multiple roles have been demonstrated for CXCR4 in human diseases such as cellular proliferative disorders, Alzheimer's disease, HIV, rheumatoid arthritis, pulmonary fibrosis, and others. For example, expression of CXCR4 and CXCL12 have been noted in several tumor types. CXCL12 is expressed by cancer-associated fibroblast (CAFs) and is often present at high levels in the tumor microenvironment (TME). In clinical studies of a wide range of tumor types, including breast, ovarian, renal, lung, and melanoma, expression of CXCR4/CXCL12 has been associated with a poor prognosis and with an increased risk of metastasis to lymph nodes, lung, liver, and brain, which are sites of CXCL12 expression. CXCR4 is frequently expressed on melanoma cells, particularly the CD133+ population that is considered to represent melanoma stem cells; in vitro experiments and murine models have demonstrated that CXCL12 is chemotactic for such cells.

Furthermore, there is now evidence implicating the CXCL12/CXCR4 axis in contributing to the loss or lack of tumor responsiveness to angiogenesis inhibitors (also referred to as “angiogenic escape”). In animal cancer models, interference with CXCR4 function has been demonstrated to alter the TME and sensitize the tumor to immune attack by multiple mechanisms such as elimination of tumor re-vascularization and increasing the ratio of CD8+ T cells to Treg cells. These effects result in significantly decreased tumor burden and increased overall survival in xenograft, syngeneic, and transgenic cancer models. See Vanharanta et al. (2013) Nat Med 19: 50-56; Gale and McColl (1999) BioEssays 21: 17-28; Highfill et al. (2014) Sci Transl Med 6: ra67; Facciabene et al. (2011) Nature 475: 226-230.

These data underscore the significant, unmet need for CXCR4 inhibitors to treat the many diseases and conditions mediated by aberrant or undesired expression of the receptor, for example in cellular proliferative disorders. While various CXCR4 inhibitors are known, there is a need for CXCR4 inhibitors with improved potency and/or other physical properties.

Many potential drugs suffer from poor absorption, distribution, metabolism and/or excretion (ADME) properties that prevent their wider use or limit their use in certain indications. A potentially attractive strategy for improving a drug's metabolic properties is deuterium enrichment. Replacing one or more hydrogen atoms with deuterium atoms may result in an improved metabolic profile and reduce unwanted degradation of the compound. Deuterium is a safe, stable, non-radioactive isotope of hydrogen. Compared to hydrogen, deuterium forms a stronger bond with carbon. In some cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over recent decades, the effects of deuterium substitution on the rate of metabolism have been reported for a very small percentage of approved drugs (see, e.g., Blake, M. I. et al., J Pharm Sci, 1975, 64:367-91; Foster, A. B., Adv Drug Res, 1985, 14:1-40 (“Foster”); Kushner, D. J. et al., Can J Physiol Pharmacol, 1999, 79-88; Fisher, M. B. et al., Curr Opin Drug Discov Devel, 2006, 9:101-09 (“Fisher”)). The results have been variable and unpredictable. For some compounds, deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem., 1991, 34, 2871-76). Many drugs have multiple sites where metabolism is possible. The site(s) where deuterium substitution is required and the extent of deuteration necessary to see an effect on metabolism, if any, will be different for each drug.

The present invention addresses the need for improved CXCR4 inhibitors and provides additional advantages.

### SUMMARY OF THE INVENTION

It has now been found that deuterium-enriched CXCR4 inhibitors, such as deuterium-enriched analogs of X4P-001 (mavorixafor), pharmaceutically acceptable salts thereof, and pharmaceutically acceptable compositions thereof, are effective for use in treating CXCR4-mediated diseases, disorders, and conditions, such as those described herein. In one aspect, the present invention provides a compound of formula I:

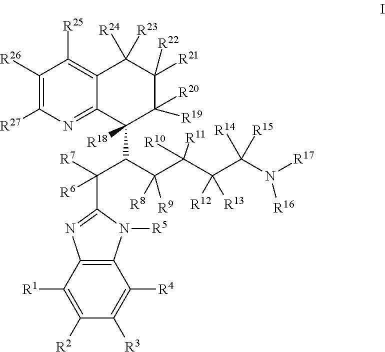

or a pharmaceutically acceptable salt thereof, wherein each of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is as defined herein.

Compounds of the present invention, and pharmaceutically acceptable compositions thereof, are useful for treating a variety of diseases, disorders, and conditions, such as those mediated by CXCR4. Such diseases, disorders, and conditions include those described herein.

### DETAILED DESCRIPTION OF THE INVENTION

### 1. General Description of Certain Aspects of the Invention

The present invention provides compounds, compositions, and methods for treatment, prevention, and/or reduction of a risk of diseases, disorders, or conditions such as those mediated by CXCR4 or a mutant thereof. In some embodiments, such compounds include those of the formulae described herein, or a pharmaceutically acceptable salt thereof, wherein each variable is as defined herein and described in embodiments.

In one aspect, the present invention provides a compound of formula I:

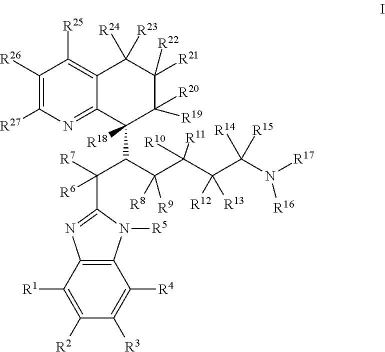

or a pharmaceutically acceptable salt thereof, wherein:
each of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is independently H or D;
provided that at least one of R¹, R², R³, R⁴, R⁵, R⁶, R⁷, R⁸, R⁹, R¹⁰, R¹¹, R, R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D.

### 2. Definitions

The term “compound” refers to a quantity of molecules that is sufficient to be weighed, tested for its structural identity, and to have a demonstrable use (e.g., a quantity that can be shown to be active in an assay, an in vitro test, or in vivo test, or a quantity that can be administered to a patient and provide a therapeutic benefit).

Deuterium (D or ²H) is a stable, non-radioactive isotope of hydrogen and has an atomic weight of 2.0144. Hydrogen naturally occurs as a mixture of the isotopes ¹H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015% should be considered unnatural and, as a result, novel, over their non-enriched counterparts.

All percentages given for the amount of deuterium present are mole percentages.

It can be quite difficult in the laboratory to achieve 100% deuteration at any one site of a lab scale amount of compound (e.g., milligram or greater). When 100% deuteration is recited or a deuterium atom is specifically shown in a structure, it is assumed that a small percentage of hydrogen may still be present. Deuterium-enrichment can be achieved by either exchanging protons with deuterium or by synthesizing the molecule with enriched starting materials.

Unless indicated otherwise, when a D is specifically recited at a position or is shown in a formula, this D represents a mixture of hydrogen and deuterium where the amount (incorporation, abundance or enrichment) of deuterium is about 50% or greater. In some embodiments, the amount (incorporation, abundance or enrichment) of deuterium is about 100% (i.e., the % incorporation of deuterium ranges from greater than 90% up to 100%). In certain embodiments, the incorporation of deuterium in D is from 95% to 100%, or from 97% to 100%. As used herein in the context of deuterium enrichment, the term “about” means±2%. In compounds provided by the present invention, any atom not designated as deuterium is present at its natural isotopic abundance, unless indicated otherwise.

The term “patient” refers to organisms to be treated by the methods of the present invention. Such organisms preferably include, but are not limited to, mammals (e.g., murines, simians, equines, bovines, porcines, canines, felines, and the like). In some embodiments, the patient is a human.

As used herein, the term “effective amount” refers to the amount of a compound sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

“Therapeutically effective amount” includes an amount of a compound of the invention that is effective when administered alone or in combination to treat the desired condition or disorder. “Therapeutically effective amount” includes an amount of a combination of compounds claimed that is effective to treat the desired condition or disorder. The combination of compounds can be additive and is preferably a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower incidence of adverse side effects and/or toxicity, increased efficacy, or some other beneficial effect of the combination compared with the individual components.

The invention provides deuterium-enriched compounds and pharmaceutically acceptable salts thereof, pharmaceutical compositions thereof, and methods of treating a disease, disorder, or condition using a deuterium-enriched compound or pharmaceutically acceptable salt thereof described herein. The deuterium-enriched compounds described herein contain deuterium enrichment at one or more positions. Deuterium enrichment at a chiral center of the compound reduces the rate at which the two enantiomers of the compound may interconvert. Further, the deuterium-enriched compounds may be provided in enantiomerically pure form. These features are contemplated to provide therapeutic agents with improved properties.

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in Organic Chemistry, Thomas Sorrell, University Science Books, Sausalito: 1999, and March's Advanced Organic Chemistry, 5^{th} Ed., Ed.: Smith, M. B. and March, J., John Wiley & Sons, New York: 2001, the entire contents of which are hereby incorporated by reference.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

### 3. Description of Exemplary Compounds

Complete deuteration, or 100% deuteration, at any one site can be difficult to achieve in the laboratory. When a deuterium atom is indicated at a given site on any compound described herein, it is understood that a small percentage of hydrogen may still be present. Such compounds are said to be enriched with deuterium. Typically, deuterium-enriched compounds are prepared via chemical synthesis utilizing appropriately enriched starting materials or by exchange of H atoms for D atoms using deuterium-containing reagents. As used herein, the terms “deuterium-enriched”or “deuterium enrichment” refer to a compound, or a particular site of said compound, which comprises deuterium in an amount that is greater than its natural isotopic abundance (0.015%). Accordingly, in some embodiments, the present invention provides compounds comprising deuterium at a given site, wherein the percentage or level of deuterium incorporation is greater than its natural isotopic abundance.

According to one aspect, the present invention provides a compound of formula I:

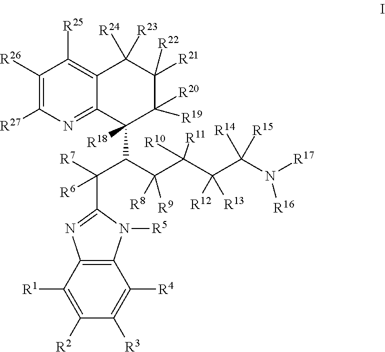

or a pharmaceutically acceptable salt thereof, wherein:
each of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is independently H or D;
provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D.

In some embodiments, at least one of R¹, R², R³, R⁴, R⁶, R⁷, R⁸, R⁹, R¹⁰, R¹¹, R¹², R¹³, R¹⁴, R¹⁵, R¹⁸, R¹⁹, R²⁰, R²¹, R²², R²³, R²⁴, R²⁵, R²⁶, and R²⁷ is D.

In some embodiments, the present invention provides a compound of Formula I-a, I-b, I-c, I-d, I-e, I-f, I-g, I-h, or I-i:

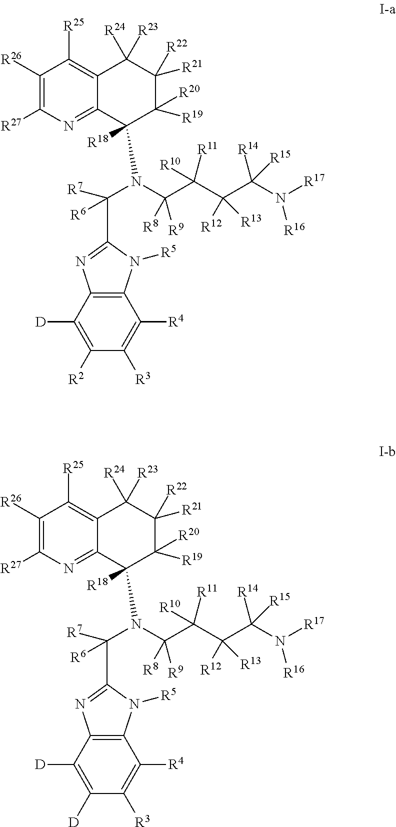

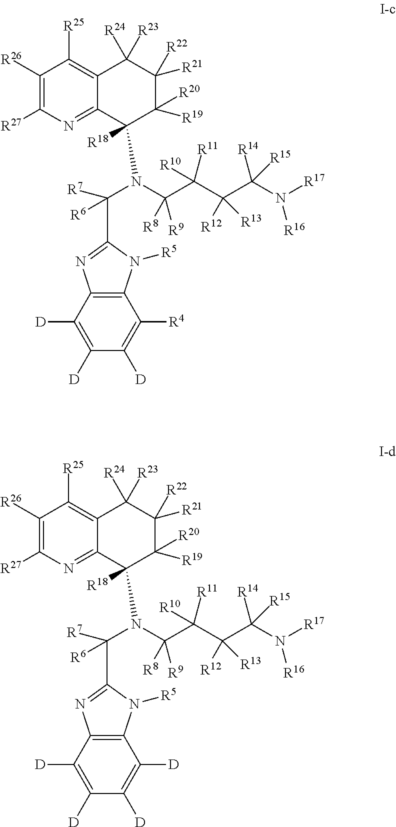

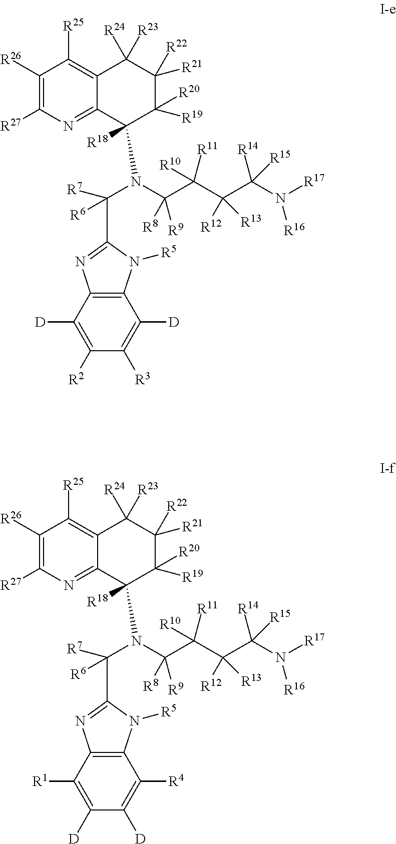

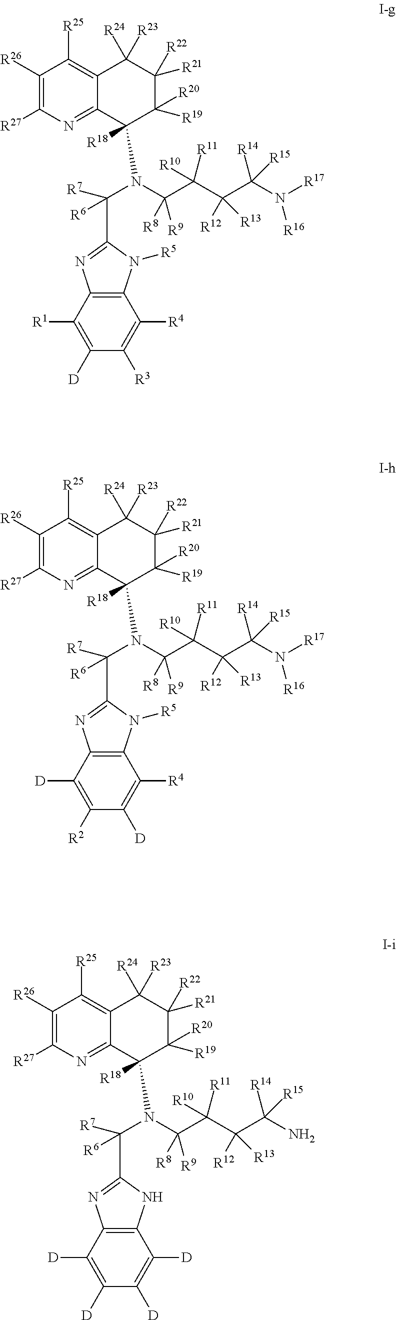

or a pharmaceutically acceptable salt thereof, wherein each variable is as defined herein, either singly or in combination.

The following embodiments are applicable to each of the preceding formulae.

As defined above and described herein, R¹ is H or D.

In some embodiments, R¹ is H and at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D. In some embodiments, R¹ is D.

As defined above and described herein, R² is H or D.

In some embodiments, R² is H and at least one of R¹, R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D. In some embodiments, R² is D.

As defined above and described herein, R³ is H or D.

In some embodiments, R³ is H and at least one of R¹, R², R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D. In some embodiments, R³ is D.

As defined above and described herein, R⁴ is H or D.

In some embodiments, R⁴ is H and at least one of R¹, R², R³, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D. In some embodiments, R⁴ is D.

As defined above and described herein, R⁵ is H or D.

In some embodiments, R⁵ is H and at least one of R¹, R², R³, R⁴, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D. In some embodiments, R⁵ is D.

As defined above and described herein, R⁶ is H or D.

In some embodiments, R⁶ is H and at least one of R¹, R², R³, R⁴, R⁵, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D. In some embodiments, R⁶ is D.

As defined above and described herein, R⁷ is H or D.

In some embodiments, R⁷ is H and at least one of R¹, R², R³, R⁴, R⁵, R⁶, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D. In some embodiments, R⁷ is D.

As defined above and described herein, R⁸ is H or D.

In some embodiments, R⁸ is D. In some embodiments, R⁸ is H and at least one of R¹, R², R³, R⁴, R⁵, R⁶, R⁷, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D.

As defined above and described herein, R⁹ is H or D.

In some embodiments, R⁹ is D. In some embodiments, R⁹ is H and at least one of R¹, R², R³, R⁴, R⁵, R⁶, R⁷, R⁸, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D.

As defined above and described herein, R¹⁰ is H or D.

In some embodiments, R¹⁰ is D. In some embodiments, R¹⁰ is H and at least one of R¹, R², R³, R⁴, R⁵, R⁶, R⁷, R⁸, R⁹, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D.

As defined above and described herein, R¹¹ is H or D.

In some embodiments, R¹¹ is D. In some embodiments, R¹¹ is H and at least one of R¹, R², R³, R⁴, R⁵, R⁶, R⁷, R⁸, R⁹, R¹⁰, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D.

As defined above and described herein, R¹² is H or D.

In some embodiments, R¹² is D. In some embodiments, R¹² is H and at least one of R¹, R², R³, R⁴, R⁵, R⁶, R⁷, R⁸, R⁹, R¹⁰, R¹¹, R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D.

As defined above and described herein, R¹³ is H or D.

In some embodiments, R¹³ is D. In some embodiments, R¹³ is H and at least one of R¹, R², R³, R⁴, R⁵, R⁶, R⁷, R⁸, R⁹, R¹⁰, R¹¹, R¹², R¹⁴, R¹⁵, R¹⁶, R¹⁷, R¹⁸, R¹⁹, R²⁰, R²¹, R²², R²³, R²⁴, R²⁵, R²⁶, and R²⁷ is D.

As defined above and described herein, R¹⁴ is H or D.

In some embodiments, R¹⁴ is D. In some embodiments, R¹⁴ is H and at least one of R¹, R², R³, R⁴, R⁵, R⁶, R⁷, R⁸, R⁹, R¹⁰, R¹¹, R¹², R¹³, R¹⁵, R¹⁶, R¹⁷, R¹⁸, R¹⁹, R²⁰, R²¹, R²², R²³, R²⁴, R²⁵, R²⁶, and R²⁷ is D.

As defined above and described herein, R¹⁵ is H or D.

In some embodiments, R¹⁵ is D. In some embodiments, R¹⁵ is H and at least one of R¹, R², R³, R⁴, R⁵, R⁶, R⁷, R⁸, R⁹, R¹⁰, R¹¹, R¹², R¹³, R¹⁴, R¹⁶, R¹⁷, R¹⁸, R¹⁹, R²⁰, R²¹, R²², R²³, R²⁴, R²⁵, R²⁶, and R²⁷ is D.

As defined above and described herein, R¹⁶ is H or D.

In some embodiments, R¹⁶ is D. In some embodiments, R¹⁶ is H and at least one of R¹, R², R³, R⁴, R⁵, R⁶, R⁷, R⁸, R⁹, R¹⁰, R¹¹, R¹², R¹³, R¹⁴, R¹⁵, R¹⁷, R¹⁸, R¹⁹, R²⁰, R²¹, R²², R²³, R²⁴, R²⁵, R²⁶, and R²⁷ is D.

As defined above and described herein, R¹⁷ is H or D.

In some embodiments, R¹⁷ is D. In some embodiments, R¹⁷ is H and at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁸, R¹⁹, R²⁰, R²¹, R²², R²³, R²⁴, R²⁵, R²⁶, and R²⁷ is D.

As defined above and described herein, R¹⁸ is H or D.

In some embodiments, R¹⁸ is D. In some embodiments, R¹⁸ is H and at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁹, R²⁰, R²¹, R²², R²³, R²⁴, R²⁵, R²⁶, and R²⁷ is D.

As defined above and described herein, R¹⁹ is H or D.

In some embodiments, R¹⁹ is D. In some embodiments, R¹⁹ is H and at least one of R¹, R², R³, R⁴, R⁵, R⁶, R⁷, Ra, R⁹, R¹⁰, R¹¹, R¹², R¹³, R¹⁴, R¹⁵, R¹⁶, R¹⁷, R¹⁸, R²⁰, R²¹, R²², R²³, R²⁴, R²⁵, R²⁶, and R²⁷ is D.

As defined above and described herein, R²⁰ is H or D.

In some embodiments, R²⁰ is D. In some embodiments, R²⁰ is H and at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²¹, R²², R²³, R²⁴, R²⁵, R²⁶, and R²⁷ is D.

As defined above and described herein, R²¹ is H or D.

In some embodiments, R²¹ is D. In some embodiments, R²¹ is H and at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²², R²³, R²⁴, R²⁵, R²⁶, and R²⁷ is D.

As defined above and described herein, R²² is H or D.

In some embodiments, R²² is D. In some embodiments, R²² is H and at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²³, R²⁴, R²⁵, R²⁶, and R²⁷ is D.

As defined above and described herein, R²³ is H or D.

In some embodiments, R²³ is D. In some embodiments, R²³ is H and at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²⁴, R²⁵, R²⁶, and R²⁷ is D.

As defined above and described herein, R²⁴ is H or D.

In some embodiments, R²⁴ is D. In some embodiments, R²⁴ is H and at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁵, R²⁶, and R²⁷ is D.

As defined above and described herein, R²⁵ is H or D.

In some embodiments, R²⁵ is D. In some embodiments, R²⁵ is H and at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁶, and R²⁷ is D.

As defined above and described herein, R²⁶ is H or D.

In some embodiments, R²⁶ is D. In some embodiments, R²⁶ is H and at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, and R²⁷ is D.

As defined above and described herein, R²⁷ is H or D.

In some embodiments, R²⁷ is D. In some embodiments, R²⁷ is H and at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, and R²⁶ is D.

In some embodiments, the present invention provides a compound of any one of Formulae I, I-a, I-b, I-c, I-d, I-e, I-f, I-g, I-h, or I-i, or a pharmaceutically acceptable salt thereof, wherein each of R¹, R², R³, and R⁴ is as defined above and described herein, and wherein each of R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶ and R²⁷ is as defined in an entry set forth in Table 1a, below.

TABLE 1a
Exemplary Deuterium-Enriched Compounds.

| Entry | R⁵ | R⁶ | R⁷ | R⁸ | R⁹ | R¹⁰ | R¹¹ | R¹² | R¹³ | R¹⁴ | R¹⁵ | R¹⁶ | R¹⁷ | R¹⁸ | R¹⁹ | R²⁰ | R²¹ | R²² | R²³ | R²⁴ | R²⁵ | R²⁶ | R²⁷ |
|---|---|---|---|---|---|---|---|---|---|---|---|---|---|---|---|---|---|---|---|---|---|---|---|
| i | D | H | H | H | H | H | H | H | H | H | H | H | H | H | H | H | H | H | H | H | H | H | H |
| ii | H | D | H | H | H | H | H | H | H | H | H | H | H | H | H | H | H | H | H | H | H | H | H |
| iii | H | H | D | H | H | H | H | H | H | H | H | H | H | H | H | H | H | H | H | H | H | H | H |
| iv | H | H | H | D | H | H | H | H | H | H | H | H | H | H | H | H | H | H | H | H | H | H | H |
| v | H | H | H | H | D | H | H | H | H | H | H | H | H | H | H | H | H | H | H | H | H | H | H |
| vi | H | H | H | H | H | D | H | H | H | H | H | H | H | H | H | H | H | H | H | H | H | H | H |
| vii | H | H | H | H | H | H | D | H | H | H | H | H | H | H | H | H | H | H | H | H | H | H | H |
| viii | H | H | H | H | H | H | H | D | H | H | H | H | H | H | H | H | H | H | H | H | H | H | H |
| ix | H | H | H | H | H | H | H | H | D | H | H | H | H | H | H | H | H | H | H | H | H | H | H |
| x | H | H | H | H | H | H | H | H | H | D | H | H | H | H | H | H | H | H | H | H | H | H | H |
| xi | H | H | H | H | H | H | H | H | H | H | D | H | H | H | H | H | H | H | H | H | H | H | H |
| xii | H | H | H | H | H | H | H | H | H | H | H | D | H | H | H | H | H | H | H | H | H | H | H |
| xiii | H | H | H | H | H | H | H | H | H | H | H | H | D | H | H | H | H | H | H | H | H | H | H |
| xiv | H | H | H | H | H | H | H | H | H | H | H | H | H | D | H | H | H | H | H | H | H | H | H |
| xv | H | H | H | H | H | H | H | H | H | H | H | H | H | H | D | H | H | H | H | H | H | H | H |
| xvi | H | H | H | H | H | H | H | H | H | H | H | H | H | H | H | D | D | H | H | H | H | H | H |
| xvii | H | H | H | H | H | H | H | H | H | H | H | H | H | H | H | H | H | D | H | H | H | H | H |
| xviii | H | H | H | H | H | H | H | H | H | H | H | H | H | H | H | H | H | H | D | H | H | H | H |
| xix | H | H | H | H | H | H | H | H | H | H | H | H | H | H | H | H | H | H | H | D | H | H | H |
| xx | H | H | H | H | H | H | H | H | H | H | H | H | H | H | H | H | H | H | H | H | D | H | H |
| xxi | H | H | H | H | H | H | H | H | H | H | H | H | H | H | H | H | H | H | H | H | H | D | H |
| xxii | H | H | H | H | H | H | H | H | H | H | H | H | H | H | H | H | H | H | H | H | H | H | D |
| xxiii | H | H | H | H | H | H | H | H | H | H | H | H | H | H | H | H | H | H | H | H | H | H | H |
| xxiv | H | D | D | H | H | H | H | H | H | H | H | H | H | H | H | H | H | H | H | H | H | H | H |
| xxv | H | H | H | D | D | H | H | H | H | H | H | H | H | H | H | H | H | H | H | H | H | H | H |
| xxvi | H | H | H | H | H | D | D | H | H | H | H | H | H | H | H | H | H | H | H | H | H | H | H |
| xxvii | H | H | H | H | H | H | H | D | D | H | H | H | H | H | H | H | H | H | H | H | H | H | H |
| xxviii | H | H | H | H | H | H | H | H | H | D | D | H | H | H | H | H | H | H | H | H | H | H | H |
| xxix | H | H | H | H | H | H | H | H | H | H | H | D | D | H | H | H | H | H | H | H | H | H | H |
| xxx | H | H | H | H | H | H | H | H | H | H | H | H | H | D | D | H | D | H | H | H | H | H | H |
| xxxi | H | H | H | H | H | H | H | H | H | H | H | H | H | H | H | D | H | H | H | H | D | D | D |
| xxxii | H | H | H | H | H | H | H | H | H | H | H | H | H | D | H | H | H | H | H | H | D | D | D |

In some embodiments, the present invention provides a compound of any one of Formulae I, I-a, I-b, I-c, I-d, I-e, I-f, I-g, I-h, or I-i, or a pharmaceutically acceptable salt thereof, wherein each of R⁵, R⁶, R⁷, R⁸, R⁹, R¹⁰, R¹¹, R¹², R¹³, R¹⁴, and R¹⁵ is as defined in an entry set forth below:

TABLE 1b
Exemplary Deuterium-Enriched Compounds.

| Entry | R⁵ | R⁶ | R⁷ | R⁸ | R⁹ | R¹⁰ | R¹¹ | R¹² | R¹³ | R¹⁴ | R¹⁵ |
|---|---|---|---|---|---|---|---|---|---|---|---|
| i | D | H | H | H | H | H | H | H | H | H | H |
| ii | H | D | H | H | H | H | H | H | H | H | H |
| iii | H | H | D | H | H | H | H | H | H | H | H |
| iv | H | H | H | D | H | H | H | H | H | H | H |
| v | H | H | H | H | D | H | H | H | H | H | H |
| vi | H | H | H | H | H | D | H | H | H | H | H |
| vii | H | H | H | H | H | H | D | H | H | H | H |
| viii | H | H | H | H | H | H | H | D | H | H | H |
| ix | H | H | H | H | H | H | H | H | D | H | H |
| x | H | H | H | H | H | H | H | H | H | D | H |
| xi | H | H | H | H | H | H | H | H | H | H | D |
| xii | H | D | H | D | H | H | H | H | H | H | H |
| xiii | H | H | D | H | D | H | H | H | H | H | H |
| xiv | H | H | H | D | H | D | H | H | H | H | H |
| xv | H | H | H | H | D | H | D | H | H | H | H |
| xvi | H | H | H | H | H | D | H | D | H | H | H |
| xvii | H | H | H | H | H | H | D | H | D | H | H |
| xviii | H | H | H | H | H | H | H | D | H | D | H |
| xix | H | H | H | H | H | H | H | H | D | H | D |
| xx | H | D | H | H | D | H | H | H | H | H | H |
| xxi | H | H | D | H | H | D | H | H | H | H | H |
| xxii | H | H | H | D | H | H | D | H | H | H | H |
| xxiii | H | H | H | H | D | H | H | D | H | H | H |
| xxiv | H | H | H | H | H | D | H | H | D | H | H |
| xxv | H | H | H | H | H | H | D | H | H | D | H |
| xxvi | H | H | H | H | H | H | H | D | H | H | D |
| xxvii | D | D | H | H | H | H | H | H | H | H | H |
| xxviii | H | D | D | H | H | H | H | H | H | H | H |
| xxix | H | H | D | D | H | H | H | H | H | H | H |
| xxx | H | H | H | D | D | H | H | H | H | H | H |
| xxxi | H | H | H | H | D | D | H | H | H | H | H |
| xxxii | H | H | H | H | H | D | D | H | H | H | H |
| xxxiii | H | H | H | H | H | H | D | D | H | H | H |
| xxxiv | H | H | H | H | H | H | H | D | D | H | H |
| xxxv | H | H | H | H | H | H | H | H | D | D | H |
| xxxvi | H | H | H | H | H | H | H | H | H | D | D |

In some embodiments, the present invention provides a compound of any one of Formulae I, I-a, I-b, I-c, I-d, I-e, I-f, I-g, I-h, or I-i, or a pharmaceutically acceptable salt thereof, wherein each of R¹⁶, R¹⁷, R¹⁸, R¹⁹, and R²⁰ is as defined in an entry set forth below:

TABLE 1c
Exemplary Deuterium-Enriched Compounds.

| Entry | R¹⁶ | R¹⁷ | R¹⁸ | R¹⁹ | R²⁰ |
|---|---|---|---|---|---|
| i | D | H | H | H | H |
| ii | H | D | H | H | H |
| iii | H | H | D | H | H |
| iv | H | H | H | D | H |
| v | H | H | H | H | D |
| vi | D | D | H | H | H |
| vii | H | D | D | H | H |
| viii | H | H | D | D | H |
| ix | H | H | H | D | D |
| x | D | H | D | H | H |
| xi | H | D | H | D | H |
| xii | H | H | D | H | D |
| xiii | D | H | H | D | H |
| xiv | H | D | H | H | D |
| xv | D | H | H | H | D |
| xvi | D | D | D | H | H |
| xvii | H | D | D | D | H |
| xviii | H | H | D | D | D |
| xix | D | H | D | D | H |
| xx | H | D | H | D | D |
| xxi | D | D | D | D | H |
| xxii | H | D | D | D | D |
| xxiii | D | H | D | D | D |
| xxiv | D | D | H | D | D |
| xxv | D | D | D | H | D |
| xxvi | D | D | D | D | D |

In some embodiments, the present invention provides a compound of any one of Formulae I, I-a, I-b, I-c, I-d, I-e, I-f, I-g, I-h, or I-i, or a pharmaceutically acceptable salt thereof, wherein each of R²¹, R²², R²³, R²⁴, R²⁵, R²⁶, and R²⁷ is as defined in an entry set forth below:

TABLE 1d
Exemplary Deuterium-Enriched
Compounds.

| Entry | R²¹ | R²² | R²³ | R²⁴ | R²⁵ | R²⁶ | R²⁷ |
|---|---|---|---|---|---|---|---|
| i | D | H | H | H | H | H | H |
| ii | H | D | H | H | H | H | H |
| iii | H | H | D | H | H | H | H |
| iv | H | H | H | D | H | H | H |
| v | H | H | H | H | D | H | H |
| vi | H | H | H | H | H | D | H |
| vii | H | H | H | H | H | H | D |
| viii | D | D | H | H | H | H | H |
| ix | H | D | D | H | H | H | H |
| x | H | H | D | D | H | H | H |
| xi | H | H | H | D | D | H | H |
| xii | H | H | H | H | D | D | H |
| xiii | H | H | H | H | H | D | D |
| xiv | D | H | D | H | H | H | H |
| xv | H | D | H | D | H | H | H |
| xvi | H | H | D | H | D | H | H |
| xvii | H | H | H | D | H | D | H |
| xviii | H | H | H | H | D | H | D |
| xix | D | H | H | D | H | H | H |
| xx | H | D | H | H | D | H | H |
| xxi | H | H | D | H | H | D | H |
| xxii | H | H | H | D | H | H | D |
| xxiii | D | H | H | H | D | H | H |
| xxiv | H | D | H | H | H | D | H |
| xxv | H | H | D | H | H | H | D |
| xxvi | D | H | H | H | H | D | H |
| xxvii | H | D | H | H | H | H | D |
| xxviii | D | H | H | H | H | H | D |
| xxix | D | D | D | H | H | H | H |
| xxx | H | D | D | D | H | H | H |
| xxxi | H | H | D | D | D | H | H |
| xxxii | H | H | H | D | D | D | H |
| xxxiii | H | H | H | H | D | D | D |

or a pharmaceutically acceptable salt thereof.

In some embodiments, the present invention provides a compound of Formula I-d,

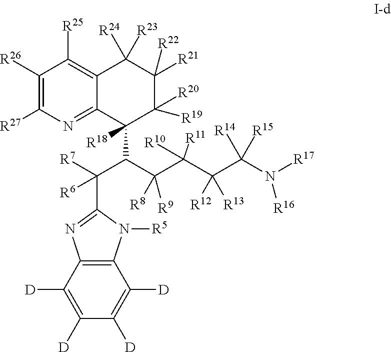

or a pharmaceutically acceptable salt thereof, wherein each of R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is as defined in an entry set forth in Table 1a, Table 1b, Table 1c, or Table 1d, above.

In some embodiments, at least one of R¹, R², R³, and R⁴ has a % deuterium incorporation of 50% or greater. In some embodiments, at least two of R¹, R², R³, and R⁴ has a % deuterium incorporation of 50% or greater. In some embodiments, at least three of R¹, R², R³, and R⁴ has a % deuterium incorporation of 50% or greater. In some embodiments, each of R¹, R², R³, and R⁴ has a % deuterium incorporation of 50% or greater.

In some embodiments, at least one of R¹, R², R³, and R⁴ has a % deuterium incorporation of 70% or greater. In some embodiments, at least two of R¹, R², R³, and R⁴ has a % deuterium incorporation of 70% or greater. In some embodiments, at least three of R¹, R², R³, and R⁴ has a % deuterium incorporation of 70% or greater. In some embodiments, each of R¹, R², R³, and R⁴ has a % deuterium incorporation of 70% or greater.

In some embodiments, at least one of R¹, R², R³, and R⁴ has a % deuterium incorporation of 80% or greater. In some embodiments, at least two of R¹, R², R³, and R⁴ has a % deuterium incorporation of 80% or greater. In some embodiments, at least three of R¹, R², R³, and R⁴ has a % deuterium incorporation of 80% or greater. In some embodiments, each of R¹, R², R³, and R⁴ has a % deuterium incorporation of 80% or greater.

In some embodiments, at least one of R¹, R², R³, and R⁴ has a % deuterium incorporation of 90% or greater. In some embodiments, at least two of R¹, R², R³, and R⁴ has a % deuterium incorporation of 90% or greater. In some embodiments, at least three of R¹, R², R³, and R⁴ has a % deuterium incorporation of 90% or greater. In some embodiments, each of R¹, R², R³, and R⁴ has a % deuterium incorporation of 90% or greater.

In some embodiments, at least one of R¹, R², R³, and R⁴ has a % deuterium incorporation of 95% or greater. In some embodiments, at least two of R¹, R², R³, and R⁴ has a % deuterium incorporation of 95% or greater. In some embodiments, at least three of R¹, R², R³, and R⁴ has a % deuterium incorporation of 95% or greater. In some embodiments, each of R¹, R², R³, and R⁴ has a % deuterium incorporation of 95% or greater.

In some embodiments, the present invention provides a deuterium-enriched compound or pharmaceutically acceptable salt thereof of Formula I, I-a, I-b, I-c, I-d, I-e, I-f, I-g, I-h, or I-i, wherein a variable indicated as D in Table 1a, Table 1b, Table 1c, or Table 1d, has a deuterium % incorporation (% abundance) of about 30% or greater. In some embodiments, the % deuterium incorporation is about 50% or greater; or about 70%, 75%, 80%, 85%, 90%, 91%, 92%, 93%, 94%, 95%, 96%, 97%, 98%, 99%, or about 99.5% or greater.

In some embodiments, the present invention provides a compound selected from those recited in Table 1a, Table 1b, Table 1c, or Table 1d, or a pharmaceutically acceptable salt thereof.

In some embodiments, present invention provides a compound of Formula I selected from these depicted in Table 2, below.

TABLE 2
Further Exemplary Deuterium-Enriched Compounds.

| | |
|---|---|
| 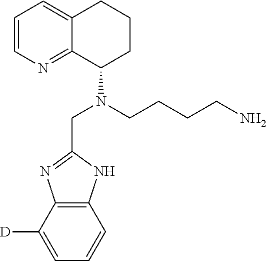 [SMILES: [2H]c1cccc2[nH]c(CN(CCCCN)[C@H]3CCCc4cccnc43)nc12] | I-1 |
| 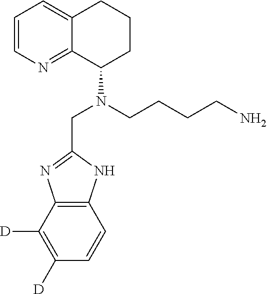 [SMILES: [2H]c1ccc2[nH]c(CN(CCCCN)[C@H]3CCCc4cccnc43)nc2c1[2H]] | I-2 |
| 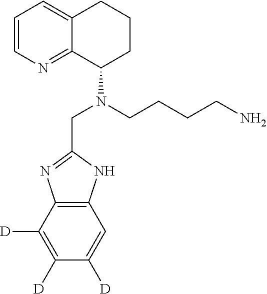 [SMILES: [2H]c1cc2[nH]c(CN(CCCCN)[C@H]3CCCc4cccnc43)nc2c([2H])c1[2H]] | I-3 |
| 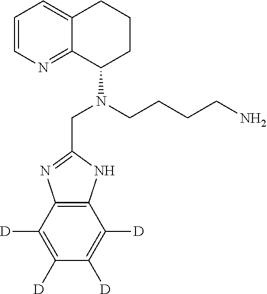 [SMILES: [2H]c1c([2H])c([2H])c2[nH]c(CN(CCCCN)[C@H]3CCCc4cccnc43)nc2c1[2H]] | I-4 |
| 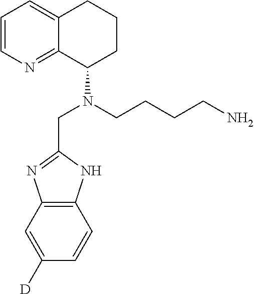 [SMILES: [2H]c1ccc2[nH]c(CN(CCCCN)[C@H]3CCCc4cccnc43)nc2c1] | I-5 |
| 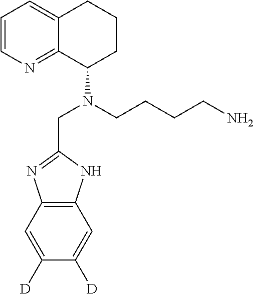 [SMILES: [2H]c1cc2nc(CN(CCCCN)[C@H]3CCCc4cccnc43)[nH]c2cc1[2H]] | I-6 |
| 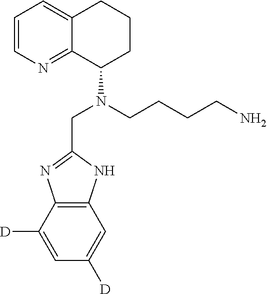 [SMILES: [2H]c1cc([2H])c2nc(CN(CCCCN)[C@H]3CCCc4cccnc43)[nH]c2c1] | I-7 |
| 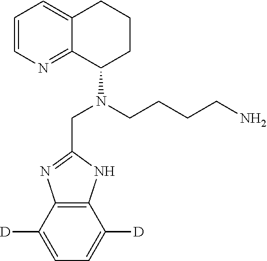 [SMILES: [2H]c1ccc([2H])c2[nH]c(CN(CCCCN)[C@H]3CCCc4cccnc43)nc12] | I-8 |
| 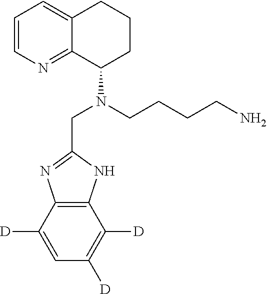 [SMILES: [2H]c1cc([2H])c2nc(CN(CCCCN)[C@H]3CCCc4cccnc43)[nH]c2c1[2H]] | I-9 |
| 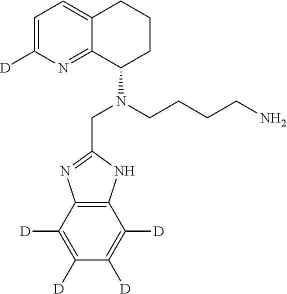 [SMILES: [2H]c1ccc2c(n1)[C@@H](N(CCCCN)Cc1nc3c([2H])c([2H])c([2H])c([2H])c3[nH]1)CCC2] | I-10 |
| 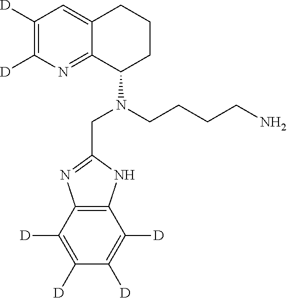 [SMILES: [2H]c1cc2c(nc1[2H])[C@@H](N(CCCCN)Cc1nc3c([2H])c([2H])c([2H])c([2H])c3[nH]1)CCC2] | I-11 |
| 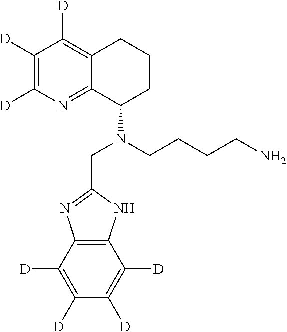 [SMILES: [2H]c1nc2c(c([2H])c1[2H])CCC[C@@H]2N(CCCCN)Cc1nc2c([2H])c([2H])c([2H])c([2H])c2[nH]1] | I-12 |
| 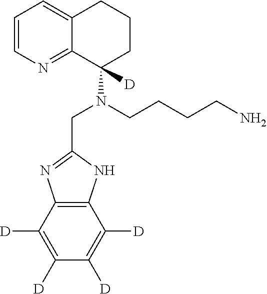 [SMILES: [2H]c1c([2H])c([2H])c2[nH]c(CN(CCCCN)[C@@]3([2H])CCCc4cccnc43)nc2c1[2H]] | I-13 |
| 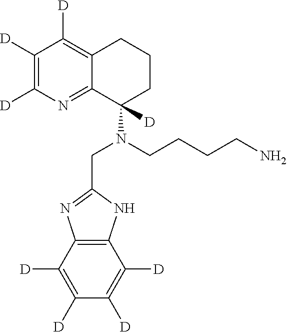 [SMILES: [2H]c1nc2c(c([2H])c1[2H])CCC[C@]2([2H])N(CCCCN)Cc1nc2c([2H])c([2H])c([2H])c([2H])c2[nH]1] | I-14 |
| 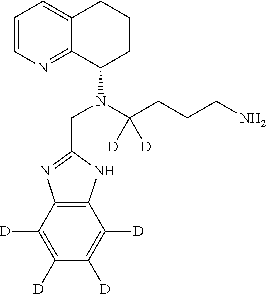 [SMILES: [2H]c1c([2H])c([2H])c2[nH]c(CN([C@H]3CCCc4cccnc43)C([2H])([2H])CCCN)nc2c1[2H]] | I-15 |
| 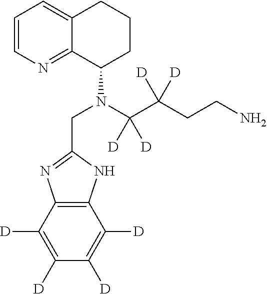 [SMILES: [2H]c1c([2H])c([2H])c2[nH]c(CN([C@H]3CCCc4cccnc43)C([2H])([2H])C([2H])([2H])CCN)nc2c1[2H]] | I-16 |
| 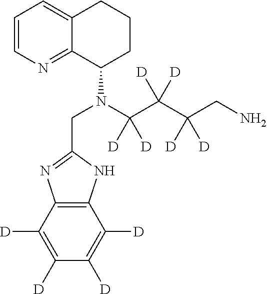 [SMILES: [2H]c1c([2H])c([2H])c2[nH]c(CN([C@H]3CCCc4cccnc43)C([2H])([2H])C([2H])([2H])C([2H])([2H])CN)nc2c1[2H]] | I-17 |
| 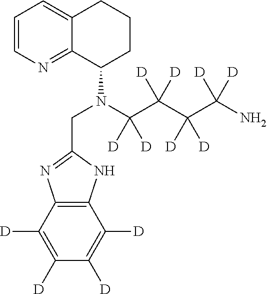 [SMILES: [2H]c1c([2H])c([2H])c2[nH]c(CN([C@H]3CCCc4cccnc43)C([2H])([2H])C([2H])([2H])C([2H])([2H])C([2H])([2H])N)nc2c1[2H]] | I-18 |
| 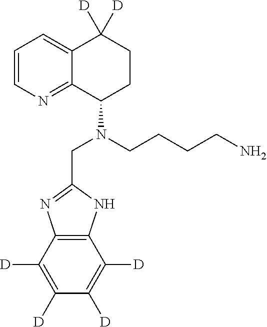 [SMILES: [2H]c1c([2H])c([2H])c2[nH]c(CN(CCCCN)[C@H]3CCC([2H])([2H])c4cccnc43)nc2c1[2H]] | I-19 |
| 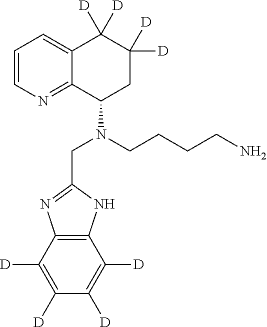 [SMILES: [2H]c1c([2H])c([2H])c2[nH]c(CN(CCCCN)[C@H]3CC([2H])([2H])C([2H])([2H])c4cccnc43)nc2c1[2H]] | I-20 |
| 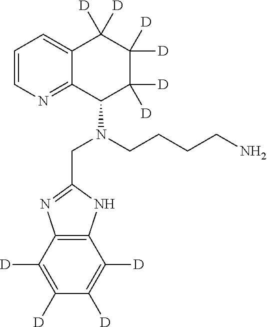 [SMILES: [2H]c1c([2H])c([2H])c2[nH]c(CN(CCCCN)[C@@H]3c4ncccc4C([2H])([2H])C([2H])([2H])C3([2H])[2H])nc2c1[2H]] | I-21 |
| 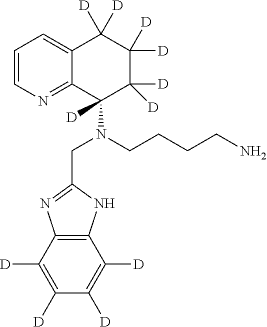 [SMILES: [2H]c1c([2H])c([2H])c2[nH]c(CN(CCCCN)[C@]3([2H])c4ncccc4C([2H])([2H])C([2H])([2H])C3([2H])[2H])nc2c1[2H]] | I-22 |
| 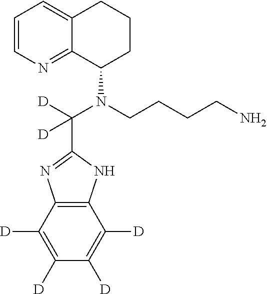 [SMILES: [2H]c1c([2H])c([2H])c2[nH]c(C([2H])([2H])N(CCCCN)[C@H]3CCCc4cccnc43)nc2c1[2H]] | I-23 |
| 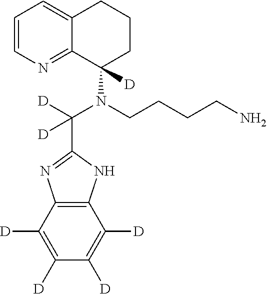 [SMILES: [2H]c1c([2H])c([2H])c2[nH]c(C([2H])([2H])N(CCCCN)[C@@]3([2H])CCCc4cccnc43)nc2c1[2H]] | I-24 |
| 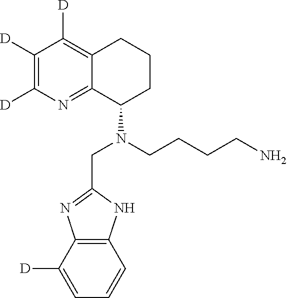 [SMILES: [2H]c1nc2c(c([2H])c1[2H])CCC[C@@H]2N(CCCCN)Cc1nc2c([2H])cccc2[nH]1] | I-25 |
| 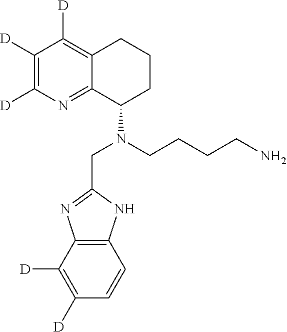 [SMILES: [2H]c1ccc2[nH]c(CN(CCCCN)[C@H]3CCCc4c3nc([2H])c([2H])c4[2H])nc2c1[2H]] | I-26 |
| 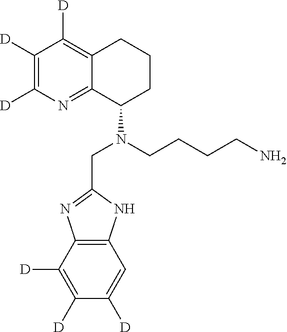 [SMILES: [2H]c1cc2[nH]c(CN(CCCCN)[C@H]3CCCc4c3nc([2H])c([2H])c4[2H])nc2c([2H])c1[2H]] | I-27 |
| 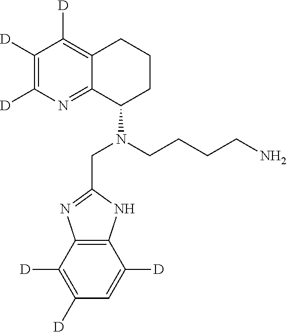 [SMILES: [2H]c1cc([2H])c2[nH]c(CN(CCCCN)[C@H]3CCCc4c3nc([2H])c([2H])c4[2H])nc2c1[2H]] | I-28 |
| 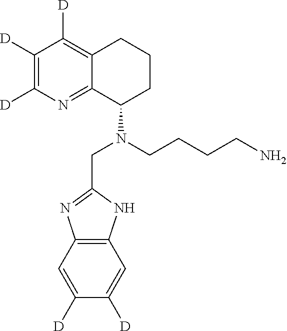 [SMILES: [2H]c1cc2nc(CN(CCCCN)[C@H]3CCCc4c3nc([2H])c([2H])c4[2H])[nH]c2cc1[2H]] | I-29 |
| 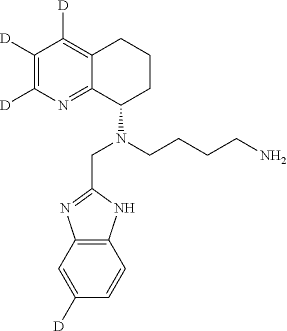 [SMILES: [2H]c1ccc2[nH]c(CN(CCCCN)[C@H]3CCCc4c3nc([2H])c([2H])c4[2H])nc2c1] | I-30 |
| 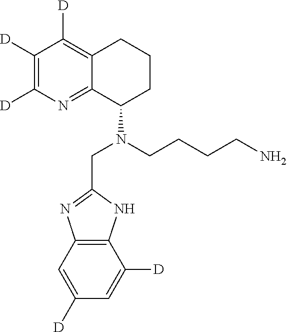 [SMILES: [2H]c1cc([2H])c2[nH]c(CN(CCCCN)[C@H]3CCCc4c3nc([2H])c([2H])c4[2H])nc2c1] | I-31 |
| 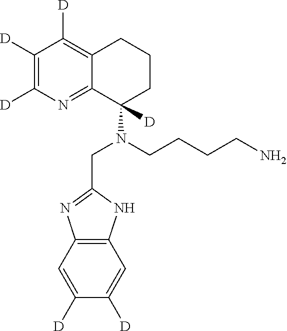 [SMILES: [2H]c1cc2nc(CN(CCCCN)[C@@]3([2H])CCCc4c3nc([2H])c([2H])c4[2H])[nH]c2cc1[2H]] | I-32 |
| 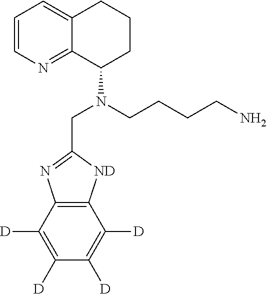 | I-33 |
| 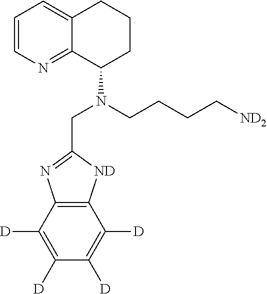 | I-34 |
| 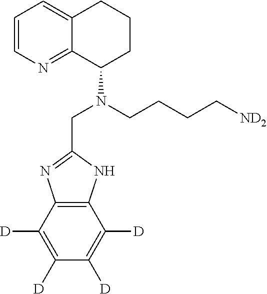 | I-35 |
| 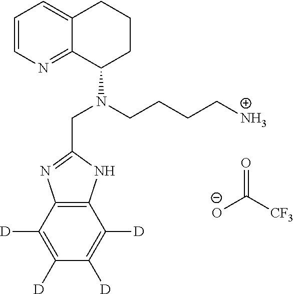 [SMILES: [2H]c1c([2H])c([2H])c2[nH]c(CN(CCCC[NH3+])[C@H]3CCCc4cccnc43)nc2c1[2H] ; O=C([O-])C(F)(F)F] | I-36 |

In some embodiments, the present invention provides a compound depicted in Table 2, above, or a pharmaceutically acceptable salt thereof.

In some embodiments, the present invention provides any compound described herein comprising one, two, three, four, five, six, seven, eight, nine, ten, eleven, twelve, thirteen, or more deuterium atoms (i.e., one or more variables in a Formula depicted herein having deuterium enrichment).

In some embodiments, each position comprising deuterium has a % deuterium incorporation in an amount of at least 5%. In some embodiments, each position comprising deuterium has a % deuterium incorporation in an amount of at least 30%. In some embodiments, each position comprising deuterium has a % deuterium incorporation in an amount of at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or at least about 99.5%.

In some embodiments, each position comprising deuterium has a % deuterium incorporation in an amount of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In some embodiments, one, two, three, four, five, six, seven, eight, nine, ten, or more positions of deuterium enrichment each independently has a % deuterium incorporation in an amount of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or about 99.5%. In some embodiments, every position of deuterium enrichment has a % deuterium incorporation in an amount of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 100%. As used herein in the context of deuterium enrichment, the term “about” means±2%.

In some embodiments, each position comprising deuterium has a % deuterium incorporation in an amount of about 5% to 99%, 10% to 95%, 15% to 90%, 20% to 85%, 25% to 80%, 30% to 80%, 35% to 75%, 40% to 70%, 30% to 99%, 30% to 95%, 30% to 90%, 50% to 75%, 50% to 99%, 50% to 95%, 50% to 90%, 60% to 99%, 60% to 95%, 60% to 90%, 70% to 99%, 70% to 95%, 70% to 90%, 80% to 99%, 80% to 95%, 80% to 90%, 90% to 99%, or 90% to 95%.

Deuterium-enriched compounds of the above Formulae may be further characterized according to the extent of deuterium enrichment (% incorporation or % abundance) at the position defined by variable R¹⁸ and/or the stereochemical purity of the compound at the position defined by variable R¹⁸. For example, in certain embodiments, the % incorporation of deuterium in R¹⁸ is at least 60%. In certain other embodiments, the % incorporation of deuterium in R¹⁸ is at least 75%. In yet other embodiments, the % incorporation of deuterium in R¹⁸ is at least 90%. In yet other embodiments, the % incorporation of deuterium in R¹⁸ is at least 95%. In yet other embodiments, the % incorporation of deuterium in R¹⁸ is from about 80% to about 99%, about 85% to about 99%, or about 90% to about 99%. In yet other embodiments, the % incorporation of deuterium in R¹⁸ is selected from: (a) at least 40%, (b) at least 50%, (c) at least 60%, (d) at least 70%, (e) at least 75%, (f) at least 80%, (g) at least 90%, (h) at least 95%, (i) at least 97%, and (j) about 100%. Additional examples of the % incorporation of deuterium in R¹⁸ include about 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, and 99 to about 100%.

In certain embodiments, the compound has a stereochemical purity of at least 85% enantiomeric excess at the carbon atom bearing variable R¹⁸. In certain other embodiments, the compound has a stereochemical purity of at least 90% enantiomeric excess at the carbon atom bearing variable R¹⁸. In certain other embodiments, the compound has a stereochemical purity of at least 95% enantiomeric excess at the carbon atom bearing variable R¹⁸. In certain other embodiments, the compound has a stereochemical purity of at least 98% enantiomeric excess at the carbon atom bearing variable R¹⁸. In certain other embodiments, the compound has a stereochemical purity of at least 99% enantiomeric excess at the carbon atom bearing variable R¹⁸. In other embodiments, the deuterium-enriched compound has a stereochemical purity of at least 80%, 85%, 90%, 95%, or 98% enantiomeric excess at a chiral carbon atom bearing variable R¹⁸, and yet additional examples of stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99% at the chiral carbon atom bearing variable R¹⁸.

### 4. Uses of Compounds and Pharmaceutically Acceptable Compositions Thereof

Certain compounds described herein are found to be useful in treating, preventing, or ameliorating various diseases, disorders, and conditions, such as those described herein.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). In some cases, treatment is continued after symptoms have resolved, for example to prevent, delay or lessen the severity of their recurrence.

The invention relates to compounds described herein for the treatment, prevention, and/or reduction of a risk of diseases, disorders, or conditions mediated by or associated with aberrant CXCR4 signalling or a mutation in CXCR4.

In one aspect, the present invention provides a method of treating a cancer, such as one of those described herein, by administering to a patient in need thereof an effective amount of a disclosed deuterium-enriched compound or a pharmaceutically acceptable salt thereof. In some embodiments, the method includes co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound or composition and the additional therapeutic agent or agents acts synergistically to prevent or reduce immune escape and/or angiogenic escape of the cancer. In some embodiments, the patient has previously been administered another anticancer agent, such as an adjuvant therapy or immunotherapy. In some embodiments, the cancer is refractory.

In some embodiments, the disease, disorder, or conditions is a hyperproliferative disorder such as cancer. In some embodiments, the cancer is breast, ovarian, renal, lung cancer, or melanoma. In some embodiments, the cancer is selected from renal cell carcinoma (RCC), refractory RCC, or clear cell RCC (ccRCC).

In some embodiments, the present invention provides a method for treating patients with cancer that presents as a solid tumor. In some embodiments, the patient has kidney cancer, renal tumor, renal carcinoma (including clear cell and papillary renal carcinoma), ovarian cancer, or melanoma.

Provided compounds are inhibitors of CXCR4 and are therefore useful for treating one or more disorders associated with activity of CXCR4. Thus, in certain embodiments, the present invention provides a method for treating a CXCR4-mediated disorder comprising the step of administering to a patient in need thereof a disclosed deuterium-enriched compound or a pharmaceutically acceptable salt thereof, or pharmaceutically acceptable composition thereof.

In one aspect, the present invention provides a method of treating cancer in a patient in need thereof, wherein said method comprises administering to said patient a disclosed deuterium-enriched compound or a pharmaceutically acceptable salt thereof in combination with one or more additional therapeutic agents, such as one or more immunostimulatory therapeutic compounds.

In some embodiments, the one or more immunostimulatory therapeutic compounds are selected from elotuzumab, mifamurtide, an agonist or activator of a toll-like receptor, or an activator of RORγt.

In some embodiments, the method further comprises administering to said patient a third therapeutic agent, such as an immune checkpoint inhibitor. In some embodiments, the method comprises administering to the patient in need thereof three therapeutic agents selected from a disclosed deuterium-enriched compound or a pharmaceutically acceptable salt thereof, an immunostimulatory therapeutic compound, and an immune checkpoint inhibitor.

In some embodiments, the immune checkpoint inhibitor is selected from nivolumab, pembrolizumab, ipilimumab, avelumab, durvalumab, atezolizumab, or pidilizumab.

In another aspect, the present invention provides a method of treating cancer in a patient in need thereof, wherein said method comprises administering to said patient a disclosed deuterium-enriched compound or a pharmaceutically acceptable salt thereof in combination with one or more additional therapeutic agents selected from an indoleamine (2,3)-dioxygenase (IDO) inhibitor, a Poly ADP ribose polymerase (PARP) inhibitor, a histone deacetylase (HDAC) inhibitor, a CDK4/CDK6 inhibitor, or a phosphatidylinositol 3 kinase (PI3K) inhibitor.

In some embodiments, the IDO inhibitor is selected from epacadostat, indoximod, capmanitib, GDC-0919, PF-06840003, BMS:F001287, Phy906/KD108, or an enzyme that breaks down kynurenine.

In some embodiments, the PARP inhibitor is selected from olaparib, rucaparib, or niraparib.

In some embodiments, the HDAC inhibitor is selected from vorinostat, romidepsin, panobinostat, belinostat, entinostat, or chidamide.

In some embodiments, the CDK 4/6 inhibitor is selected from palbociclib, ribociclib, abemaciclib or trilaciclib.

In some embodiments, the method further comprises administering to said patient a third therapeutic agent, such as an immune checkpoint inhibitor. In some embodiments, the method comprises administering to the patient in need thereof three therapeutic agents selected from a disclosed deuterium-enriched compound or a pharmaceutically acceptable salt thereof, a second therapeutic agent selected from an indoleamine (2,3)-dioxygenase (IDO) inhibitor, a Poly ADP ribose polymerase (PARP) inhibitor, a histone deacetylase (HDAC) inhibitor, a CDK4/CDK6 inhibitor, or a phosphatidylinositol 3 kinase (PI3K) inhibitor, and a third therapeutic agent selected from an immune checkpoint inhibitor. In some embodiments, the immune checkpoint inhibitor is selected from nivolumab, pembrolizumab, ipilimumab, avelumab, durvalumab, atezolizumab, or pidilizumab.

In some embodiments, the PI3K inhibitor is selected from idelalisib, alpelisib, taselisib, pictilisib, copanlisib, duvelisib, PQR309, or TGR1202.

In another aspect, the present invention provides a method of treating cancer in a patient in need thereof, wherein said method comprises administering to said patient a disclosed deuterium-enriched compound or a pharmaceutically acceptable salt thereof in combination with one or more additional therapeutic agents selected from a platinum-based therapeutic, a taxane, a nucleoside inhibitor, or a therapeutic agent that interferes with normal DNA synthesis, protein synthesis, cell replication, or will otherwise inhibit rapidly proliferating cells.

In some embodiments, the platinum-based therapeutic is selected from cisplatin, carboplatin, oxaliplatin, nedaplatin, picoplatin, or satraplatin.

In some embodiments, the taxane is selected from paclitaxel, docetaxel, albumin-bound paclitaxel, cabazitaxel, or SID530.

In some embodiments, the therapeutic agent that interferes with normal DNA synthesis, protein synthesis, cell replication, or will otherwise interfere with the replication of rapidly proliferating cells is selected from trabectedin, mechlorethamine, vincristine, temozolomide, cytarabine, lomustine, azacitidine, omacetaxine mepesuccinate, asparaginase Erwinia chrysanthemi, eribulin mesylate, capacetrine, bendamustine, ixabepilone, nelarabine, clorafabine, trifluridine, or tipiracil.

In some embodiments, the method further comprises administering to said patient a third therapeutic agent, such as an immune checkpoint inhibitor. In some embodiments, the method comprises administering to the patient in need thereof three therapeutic agents selected from a disclosed deuterium-enriched compound or a pharmaceutically acceptable salt thereof, a second therapeutic agent selected from a platinum-based therapeutic, a taxane, a nucleoside inhibitor, or a therapeutic agent that interferes with normal DNA synthesis, protein synthesis, cell replication, or will otherwise inhibit rapidly proliferating cells, and a third therapeutic agent selected from an immune checkpoint inhibitor.

In some embodiments, the immune checkpoint inhibitor is selected from nivolumab, pembrolizumab, ipilimumab, avelumab, durvalumab, atezolizumab, or pidilizumab.

In some embodiments, any one of the foregoing methods further comprises the step of obtaining a biological sample from the patient and measuring the amount of a disease-related biomarker.

In some embodiments, the biological sample is a blood sample.

In some embodiments, the disease-related biomarker is selected from circulating CD8+ T cells or the ratio of CD8+ T cells:Treg cells.

In some embodiments, the cancer is selected from hepatocellular carcinoma, ovarian cancer, ovarian epithelial cancer, fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the present invention provides a method for treating a cancer that presents as a solid tumor, such as a sarcoma, carcinoma, or lymphoma, comprising the step of administering a disclosed deuterium-enriched compound or a pharmaceutically acceptable salt thereof to a patient in need thereof. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, the present invention provides a method for treating a cancer selected from leukemia or a cancer of the blood, comprising administering to a patient in need thereof an effective amount of a disclosed deuterium-enriched compound or a pharmaceutically acceptable salt thereof, optionally in combination with an additional therapeutic agent such as those described herein. In some embodiments, the cancer is selected from acute myeloid leukemia (AML), chronic myeloid leukemia (CML), acute lymphocytic leukemia (ALL), chronic lymphocytic leukemia (CLL), or a virally induced leukemia.

In some embodiments, the patient has a resectable solid tumor, meaning that the patient's tumor is deemed susceptible to being removed by surgery. In other embodiments, the patient has an unresectable solid tumor, meaning that the patient's tumor has been deemed not susceptible to being removed by surgery, in whole or in part.

In some embodiments, the cancer is an advanced cancer, such as an advanced kidney cancer or advanced renal cell carcinoma.

In some embodiments, the present invention provides a method for treating refractory cancer in a patient in need thereof comprising administering to a patient in need thereof an effective amount of a disclosed deuterium-enriched compound or a pharmaceutically acceptable salt thereof or pharmaceutical composition thereof, optionally in combination with an additional therapeutic agent such as those described herein.

In certain embodiments, the patient was previously administered a protein kinase inhibitor. In some embodiments, the patient was previously administered a VEGF-R antagonist. In certain embodiments, the patient was previously administered an immune checkpoint inhibitor. In some embodiments, the patient was previously administered an immune checkpoint inhibitor selected from nivolumab (Opdivo®, Bristol-Myers Squibb), pembrolizumab (Keytruda®, Merck), or ipilumumab (Yervoy®, Bristol-Myers Squibb).

In some embodiments, a disclosed deuterium-enriched compound or a pharmaceutically acceptable salt thereof is administered to a patient in a fasted state.

### Cellular Proliferative Disorders

The present invention features methods and compositions for the diagnosis and prognosis of cellular proliferative disorders (e.g., cancer) and the treatment of these disorders by targeting CXCR4. Cellular proliferative disorders described herein include, e.g., cancer, obesity, and proliferation-dependent diseases. Such disorders may be diagnosed using methods known in the art.

### Cancer

Cancer includes, in one embodiment, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

The present invention further features methods and compositions for the diagnosis, prognosis and treatment of viral-associated cancers, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892)

In some embodiments, the present invention provides a method for treating a tumor in a patient in need thereof, comprising administering to the patient a disclosed deuterium-enriched compound or a pharmaceutically acceptable salt thereof. In some embodiments, the tumor comprises any of the cancers described herein. In some embodiments, the tumor comprises melanoma cancer. In some embodiments, the tumor comprises breast cancer. In some embodiments, the tumor comprises lung cancer. In some embodiments the the tumor comprises small cell lung cancer (SCLC). In some embodiments the the tumor comprises non-small cell lung cancer (NSCLC).

In some embodiments, the tumor is treated by arresting further growth of the tumor. In some embodiments, the tumor is treated by reducing the size (e.g., volume or mass) of the tumor by at least 5%, 10%, 25%, 50%, 75%, 90% or 99% relative to the size of the tumor prior to treatment. In some embodiments, tumors are treated by reducing the quantity of the tumors in the patient by at least 5%, 10%, 25%, 50%, 75%, 90% or 99% relative to the quantity of tumors prior to treatment.

### Primary Immune Deficiencies

In some embodiments, the present invention provides a method for treating one or more disorders, diseases, and/or conditions wherein the disorder, disease, or condition includes, but is not limited to, a primary immunodeficiency disease or disorder, comprising administering to a patient in need thereof an effective amount of a disclosed compound. Primary immune deficiencies treatable by the methods of the present invention include: warts, hypogammaglobulinemia, infections, myelokathexis (WHIM) syndrome; severe congenital neutropenia (SCN), especially those arising from G6PC3 deficiency (McDermott et al. (2010) Blood 116:2793-2802); GATA2 deficiency (Mono MAC syndrome) (Maciejweski-Duval et al. (2015) J. Leukoc. Biol. 5MA0815-288R (Epub. ahead of printing); idiopathic CD4+T lymphocytopenia (ICL); and Wiskott-Aldrich Syndrome.

In other embodiments, the invention relates to a method of inhibiting CXCR4 activity in a biological sample comprising the step of contacting said biological sample with a disclosed deuterium-enriched compound or a pharmaceutically acceptable salt thereof.

According to another embodiment, the invention relates to a method of inhibiting CXCR4, or a mutant thereof, activity in a biological sample comprising the step of contacting said biological sample with a disclosed deuterium-enriched compound or a pharmaceutically acceptable salt thereof. In certain embodiments, the invention relates to a method of inhibiting CXCR4, or a mutant thereof, activity in a biological sample comprising the step of contacting said biological sample with a disclosed deuterium-enriched compound or a pharmaceutically acceptable salt thereof.

Co-Administration with Additional Therapeutic Agents

In one aspect, the present invention provides a method of treating a cancer, such as those described herein, by administering to a patient in need thereof a disclosed deuterium-enriched compound or a pharmaceutically acceptable salt thereof. In some embodiments, the method includes co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of a disclosed deuterium-enriched compound or a pharmaceutically acceptable salt thereof and the additional therapeutic agent or agents acts synergistically to prevent or reduce immune escape and/or angiogenic escape of the cancer. In some embodiments, the patient has previously been administered another anticancer agent, such as an adjuvant therapy or immunotherapy. In some embodiments, the cancer is refractory.

Certain methods of treating a disease or disorder by co-administering X4P-001 with one or more additional agents are described in PCT Application No. PCT/US2018/038776, filed Jun. 21, 2018, the entirety of which is hereby incorporated by reference.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

In some embodiments, the additional therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TK1258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In some embodiments, the additional therapeutic agent is an mTOR inhibitor, which inhibits cell proliferation, angiogenesis and glucose uptake. Approved mTOR inhibitors useful in the present invention include everolimus (Afinitor®, Novartis); temsirolimus (Torisel®, Pfizer); and sirolimus (Rapamune®, Pfizer).

In some embodiments, the additional therapeutic agent is a Poly ADP ribose polymerase (PARP) inhibitor. Approved PARP inhibitors useful in the present invention include olaparib (Lynparza®, AstraZeneca); rucaparib (Rubraca®, Clovis Oncology); and niraparib (Zejula®, Tesaro). Other PARP inhibitors being studied which may be used in the present invention include talazoparib (MDV3800/BMN 673/LT00673, Medivation/Pfizer/Biomarin); veliparib (ABT-888, AbbVie); and BGB-290 (BeiGene, Inc.).

In some embodiments, the additional therapeutic agent is a phosphatidylinositol 3 kinase (PI3K) inhibitor. Approved PI3K inhibitors useful in the present invention include idelalisib (Zydelig®, Gilead). Other PI3K inhibitors being studied which may be used in the present invention include alpelisib (BYL719, Novartis); taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In some embodiments, the additional therapeutic agent is a proteasome inhibitor. Approved proteasome inhibitors useful in the present invention include bortezomib (Velcade®, Takeda); carfilzomib (Kyprolis®, Amgen); and ixazomib (Ninlaro®, Takeda).

In some embodiments, the additional therapeutic agent is a histone deacetylase (HDAC) inhibitor. Approved HDAC inhibitors useful in the present invention include vorinostat (Zolinza®, Merck); romidepsin (Istodax®, Celgene); panobinostat (Farydak®, Novartis); and belinostat (Beleodaq®, Spectrum Pharmaceuticals). Other HDAC inhibitors being studied which may be used in the present invention include entinostat (SNDX-275, Syndax Pharmaceuticals) (NCT00866333); and chidamide (Epidaza®, HBI-8000, Chipscreen Biosciences, China).

In some embodiments, the additional therapeutic agent is a CDK inhibitor, such as a CDK 4/6 inhibitor. Approved CDK 4/6 inhibitors useful in the present invention include palbociclib (Ibrance®, Pfizer); and ribociclib (Kisqali®, Novartis). Other CDK 4/6 inhibitors being studied which may be used in the present invention include abemaciclib (Ly2835219, Eli Lilly); and trilaciclib (G1T28, G1 Therapeutics).

In some embodiments, the additional therapeutic agent is an indoleamine (2,3)-dioxygenase (IDO) inhibitor. IDO inhibitors being studied which may be used in the present invention include epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); and an enzyme that breaks down kynurenine (Kynase, Kyn Therapeutics).

In some embodiments, the additional therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

In some embodiments, the additional therapeutic agent is an aromatase inhibitor. Approved aromatase inhibitors which may be used in the present invention include exemestane (Aromasin®, Pfizer); anastazole (Arimidex®, AstraZeneca) and letrozole (Femara®, Novartis).

In some embodiments, the additional therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, the additional therapeutic agent is a folic acid inhibitor. Approved folic acid inhibitors useful in the present invention include pemetrexed (Alimta®, Eli Lilly).

In some embodiments, the additional therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, the additional therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, the additional therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, the additional therapeutic agent is a glutaminase inhibitor. Glutaminase inhibitors being studied which may be used in the present invention include CB-839 (Calithera Biosciences).

In some embodiments, the additional therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, the additional therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, the additional therapeutic agent is a nucleoside inhibitor, or other therapeutic that interfere with normal DNA synthesis, protein synthesis, cell replication, or will otherwise inhibit rapidly proliferating cells. Such nucleoside inhibitors or other therapeutics include trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, the additional therapeutic agent is a platinum-based therapeutic, also referred to as platins. Platins cause cross-linking of DNA, such that they inhibit DNA repair and/or DNA synthesis, mostly in rapidly reproducing cells, such as cancer cells. Approved platinum-based therapeutics which may be used in the present invention include cisplatin (Platinol®, Bristol-Myers Squibb); carboplatin (Paraplatin®, Bristol-Myers Squibb; also, Teva; Pfizer); oxaliplatin (Eloxitin® Sanofi-Aventis); and nedaplatin (Aqupla®, Shionogi). Other platinum-based therapeutics which have undergone clinical testing and may be used in the present invention include picoplatin (Poniard Pharmaceuticals); and satraplatin (JM-216, Agennix).

In some embodiments, the additional therapeutic agent is a taxane compound, which causes disruption of microtubules, which are essential for cell division. Approved taxane compounds which may be used in the present invention include paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), and cabazitaxel (Jevtana®, Sanofi-Aventis). Other taxane compounds which have undergone clinical testing and may be used in the present invention include SID530 (SK Chemicals, Co.) (NCT00931008).

In some embodiments, the additional therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signalling processes should proceed.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PDl, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PDl, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, T6, and memory CD8+(ap) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-Ll monoclonal Antibody (Anti-B7-H1; MEDI4736), MK-3475 (PD-1 blocker), nivolumab (Opdivo®, BMS-936558; anti-PDl antibody), CT-011 (anti-PDl antibody), BY55 monoclonal antibody, AMP224 (anti-PDLl antibody), BMS-936559 (anti-PDLl antibody), MPLDL3280A (anti-PDLl antibody), MSB0010718C (anti-PDLl antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, or a CTLA-4 antagonist. In some embodiments, a CXCR4 antagonist such as X4P-001 or a pharmaceutically acceptable salt thereof is administered in combination with nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); or atezolizumab (anti-PD-L1 antibody, Tecentriq®, Genentech).

Other immune checkpoint inhibitors suitable for use in the present invention include REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; and PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

Nivolumab (Opdivo®, BMS-93568/MDX1106; Bristol-Myers Squibb), is a fully human IgG4 monoclonal antibody that acts as an immunomodulator by binding to the programmed cell death 1 (PD-1) receptor and selectively blocking interaction with its ligands PD-L1 and PD-L2. The structure and other properties of nivolumab are specified at http://www.drugbank.ca/drugs/DB09035, accessed on Mar. 14, 2016, the disclosure of which is hereby incorporated herein. Nivolumab is approved for use in treatment of patients with advanced renal cell carcinoma who have received prior anti-angiogenic therapy; as a single agent in certain types of unresectable or metastatic melanoma; in treating unresectable or metastatic melanoma or in combination with ipilimumab in treating unresectable or metastatic melanoma; and for treatment of metastatic non-small cell lung cancer and progression on or after platinum-based chemotherapy. Additionally, nivolumab has been tested or mentioned as a possible treatment in other oncologic indications, including solid tumors; skin melanoma; glioblastoma; glioma; gliosarcoma; astrocytoma; brain cancer; leukemia; acute myeloid leukemia; chronic myeloid leukemia; chronic lymphocytic leukemia; advanced liver cancer or hepatocellular carcinoma; uveal melanoma; prostate cancer; pancreatic neoplasm and pancreatic cancer; bladder cancer; colorectal cancer; myelodysplastic syndrome; Hodgkin Lymphoma; Non-Hodgkin Lymphoma; multiple myeloma; cervical cancer; endometrial cancer; uterine cancer; ovarian cancer and ovarian carcinoma; peritoneal carcinoma; head and neck squamous cell cancer; gastric cancer; esophageal cancer; Kaposi sarcoma; breast neoplasm, breast adenocarcinoma and breast cancer; bone sarcoma; soft tissue sarcoma; meningiomas; and mesothelioma.

In some embodiments, the present invention provides a method of treating advanced clear-cell renal cell carcinoma, comprising administering to a patient in need thereof an effective amount of a disclosed deuterium-enriched compound or a pharmaceutically acceptable salt thereof or pharmaceutical composition thereof in combination with nivolumab or everolimus, optionally wherein that patient has received previous treatment with a regimen of antiangiogenic therapy.

Generally, the amount of nivolumab or other immune checkpoint inhibitor useful in the present invention will be dependent upon the size, weight, age and condition of the patient being treated, the severity of the disorder or condition, and the discretion of the prescribing physician. For example, in its current prescribed labeling for unresectable or metastatic renal cell carcinoma, the recommended course of administration for nivolumab is 3 mg/kg as an intravenous infusion over 60 minutes every two weeks, until disease progression or unacceptable toxicity. In the discretion of the clinician, depending upon individual tolerance, the prescribed dose of nivolumab may be increased, for example, increased in dosage and/or frequency. In the discretion of the clinician, together with the warnings provided with prescribing information, administration of nivolumab may be discontinued, or the dose reduced in the case of significant adverse effects. In some embodiments, nivolumab is administered in the methods of the present invention according to the labeling guidelines above.

In some embodiments, the present invention provides a method for treating a patient by administering a CXCR4 antagonist such as X4P-001 or a pharmaceutically acceptable salt thereof in combination with an immunostimulatory therapeutics. Approved immunostimulatory therapeutics which may be used in the present invention include elotuzumab (anti-SLAMF7-antibody, Empliciti®, Bristol-Myers Squibb). Immunostimulatory compounds being studied that may be used in the present invention include mifamurtide (Mepact®, Takeda Oncology).

Another immunostimulatory therapeutic that may be used in the present invention is recombinant human interleukin 15 (rhIL-15). rhIL-15 has been tested in the clinic as a therapy for melanoma and renal cell carcinoma (NCT01021059 and NCT01369888) and leukemias (NCT02689453). Another immunostimulatory therapeutic that may be used in the present invention is recombinant human interleukin 12 (rhIL-12). Another suitable IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). Recombinant human interleukin 12 (rhIL-12) has been tested in the clinic for many oncological indications, for example, as a therapy for lymphoma (NM-IL-12, Neumedicines, Inc.), (NCT02544724 and NCT02542124).

Another paradigm for immune-stimulation is the use of oncolytic viruses. In some embodiments, the present invention provides a method for treating a patient by administering a disclosed deuterium-enriched compound or a pharmaceutically acceptable salt thereof in combination with an immunostimulatory therapy such as oncolytic viruses. Approved immunostimulatory oncolytic viruses which may be used in the present invention include talimogene laherparepvec (live, attenuated herpes simplex virus, Imlygic®, Amgen).

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™.Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, C1-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms,” such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MM1270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; Zd₆474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from a disclosed deuterium-enriched compound or a pharmaceutically acceptable salt thereof, as part of a multiple dosage regimen. Alternatively, those agents may be part of a single dosage form, mixed together with a disclosed deuterium-enriched compound or a pharmaceutically acceptable salt thereof in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both a disclosed deuterium-enriched compound or a pharmaceutically acceptable salt thereof and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed X4P-001 compositions will range from about 50% to 100% of the amount normally present in a composition comprising that agent as the only therapeutically active agent.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

International Patent Application No. PCT/US2016/066634 describes additional compositions, methods of use, and combination therapies of X4P-001, and is incorporated by reference in its entirety. Such compositions, methods of use, and combination therapies are applicable to the disclosed deuterium-enriched compounds.

### 5. Pharmaceutically Acceptable Compositions

In one aspect, the present invention provides a pharmaceutical composition comprising a disclosed compound or a pharmaceutically acceptable salt thereof, or a pharmaceutically acceptable derivative thereof, and a pharmaceutically acceptable excipient, carrier, adjuvant, or vehicle. The amount of compound in compositions of this invention is such that it is effective to measurably inhibit CXCR4, or a mutant thereof, in a biological sample or in a patient. In some embodiments, the amount of compound or a pharmaceutically acceptable salt thereof is an amount effective to treat, ameliorate, prevent, or lessen the severity of a disease, disorder, or condition, such as those described herein. In certain embodiments, a composition of this invention is formulated for administration to a patient in need of such composition. In some embodiments, a composition of this invention is formulated for oral administration to a patient.

The term “subject” or “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “inhibitorily active metabolite or residue thereof” means that a metabolite or residue thereof is also an inhibitor of CXCR4, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg or, e.g., from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

Disclosed compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In certain embodiments, the composition is formulated for oral administration in the form of a tablet or a capsule. In some embodiments, the composition comprising the deuterium-enriched CXCR4 inhibitor is formulated for oral administration in the form of a capsule.

In certain embodiments, a provided method comprises administering to the patient one or more capsules comprising 10 mg to 1200 mg deuterium-enriched mavorixafor active ingredient; and one or more pharmaceutically acceptable excipients. In certain embodiments, the capsule is comprised of hard gelatin.

In certain embodiments, the present invention provides a pharmaceutical composition comprising a deuterium-enriched compound described herein, one or more diluents, a disintegrant, a lubricant, a flow aid, and a wetting agent. In some embodiments, the present invention provides a pharmaceutical composition comprising 10 mg to 1200 mg of a deuterium-enriched compound described herein, microcrystalline cellulose, dibasic calcium phosphate dihydrate, croscarmellose sodium, sodium stearyl fumarate, colloidal silicon dioxide, and sodium lauryl sulfate. In some embodiments, the present invention provides a unit dosage form wherein said unit dosage form comprises a pharmaceutical composition comprising 10-200 mg of a deuterium-enriched compound described herein, microcrystalline cellulose, dibasic calcium phosphate dihydrate, croscarmellose sodium, sodium stearyl fumarate, colloidal silicon dioxide, and sodium lauryl sulfate. In certain embodiments, the present invention provides a unit dosage form comprising a pharmaceutical composition comprising a deuterium-enriched compound described herein such as deuterium-enriched mavorixafor, present in an amount of about 10 mg, about 20 mg, about 25 mg, about 50 mg, about 75 mg, about 100 mg, about 150 mg, about 200 mg, about 250 mg, about 300 mg, about 400 mg, about 450 mg, about 500 mg, about 600 mg, about 700 mg, about 750 mg, about 800 mg, about 900 mg, about 1000 mg, about 1100 mg, or about 1200 mg. In some embodiments, a provided composition (or unit dosage form) is administered to the patient once per day, twice per day, three times per day, or four times per day. In some embodiments, a provided composition (or unit dosage form) is administered to the patient once per day or twice per day. In some embodiments, the composition or unit dosage form is administered once per day, optionally wherein the patient is in a fasted state.

In some embodiments, the present invention provides a disclosed pharmaceutical composition in combination with an additional therapeutic agent.

In another aspect, the present invention provides a unit dosage form comprising a pharmaceutical composition comprising:
(a) a disclosed compound or pharmaceutically acceptable salt thereof, as about 10-20% by weight of the composition;
(b) microcrystalline cellulose as about 70-85% by weight of the composition;
(c) croscarmellose sodium as about 5-10% by weight of the composition;
(d) sodium stearyl fumarate as about 0.5-2% by weight of the composition; and
(e) colloidal silicon dioxide as about 0.1-1.0% by weight of the composition.

In another aspect, the present invention provides a unit dosage form comprising a pharmaceutical composition comprising:
(a) a disclosed compound or pharmaceutically acceptable salt thereof, as about 30-40% by weight of the composition;
(b) microcrystalline cellulose as about 20-25% by weight of the composition;
(c) dibasic calcium phosphate dihydrate as about 30-35% by weight of the composition;
(d) croscarmellose sodium as about 5-10% by weight of the composition;
(e) sodium stearyl fumarate as about 0.5-2% by weight of the composition;
(f) colloidal silicon dioxide as about 0.1-1.0% by weight of the composition; and
(g) sodium lauryl sulfate as about 0.1-1.0% by weight of the composition.

In another aspect, the present invention provides a unit dosage form comprising a pharmaceutical composition comprising:
(a) a disclosed compound or pharmaceutically acceptable salt thereof, as about 35-75% by weight of the composition;
(b) microcrystalline cellulose as about 5-28% by weight of the composition;
(c) dibasic calcium phosphate dihydrate as about 7-30% by weight of the composition;
(d) croscarmellose sodium as about 2-10% by weight of the composition;
(e) sodium stearyl fumarate as about 0.3-2.5% by weight of the composition;
(f) colloidal silicon dioxide as about 0.05-1.2% by weight of the composition; and
(g) sodium lauryl sulfate as about 0.2-1.2% by weight of the composition.
In some embodiments, the unit dosage form is in the form of a capsule.

In some embodiments, the capsule comprises about 25 mg, about 50 mg, about 100 mg, about 200, about 250, about 400, or about 500 mg of a disclosed deuterium-enriched analog of mavorixafor, or a pharmaceutically acceptable salt thereof.

In some embodiments, the capsule comprises about 25-150 mg, about 100-600 mg, about 200-600 mg, about 300-800 mg, about 100-800 mg, or about 200-800 mg of a disclosed deuterium-enriched analog of mavorixafor, or a pharmaceutically acceptable salt thereof.

In another aspect, the present invention provides a method of treating, preventing, or reducing a risk of a disease, disorder, or condition associated with CXCR4 in a subject in need thereof, comprising administering to the subject a disclosed compound or pharmaceutically acceptable salt thereof, or a pharmaceutical composition thereof.

Inasmuch as it may be desirable to administer a combination of active compounds, for example, for the purpose of treating a particular disease or condition, it is within the scope of the present invention that two or more pharmaceutical compositions, at least one of which contains a composition in accordance with the invention, may conveniently be combined in the form of a kit suitable for co-administration of the compositions. Thus the kit of the invention includes two or more separate pharmaceutical compositions, at least one of which contains an X4P-001 composition of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

In order that the invention described herein may be more fully understood, the following examples are set forth. It should be understood that these examples are for illustrative purposes only and are not to be construed as limiting this invention in any manner.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1. Methods of Preparation of Deuterium-Enriched Compounds

As depicted in this and other Examples below, in certain exemplary embodiments, compounds are prepared according to the following general procedures. It will be appreciated that, although the general methods depict the synthesis of particular compounds of the present invention, the following general methods, and other methods known to one of ordinary skill in the art, can be applied to the full scope of compounds described herein.

Methods of preparation applicable to certain compounds of the invention are disclosed in Crawford et al. (2008) Org. Process Res. Dev. 12:823-830; U.S. Pat. No. 7,354,934, WO 00/56729, U.S. Ser. No. 60/232,891, and U.S. Ser. No. 60/234,510, as well as An, H.; Wang, T.; Mohan, V.; Griffey, R. H.; Cook, P. D. Tetrahedron 1998, 54, 3999-4012; and U.S. Pat. No. 10,548,889; the entirety of each of which is hereby incorporated by reference. One of ordinary skill in the art is capable of varying such disclosed methods, using no more than routine experimentation, to provide alternate means of preparation, testing, and analysis of compounds of the invention.

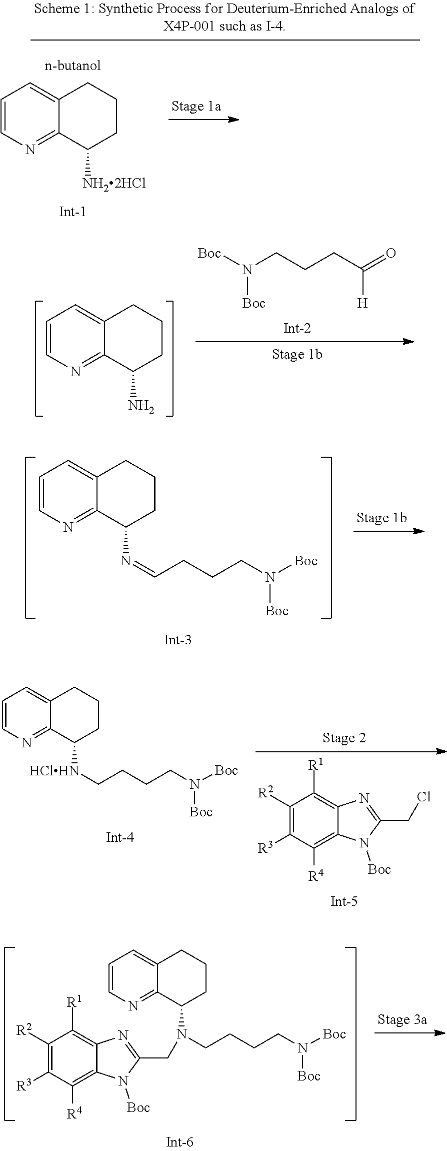

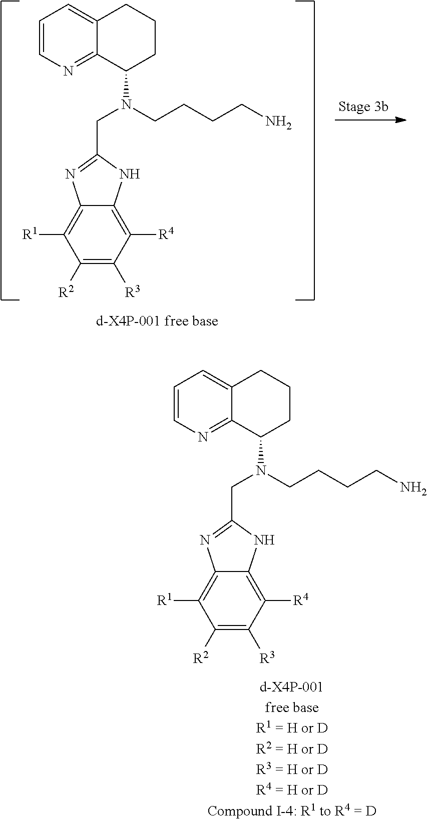

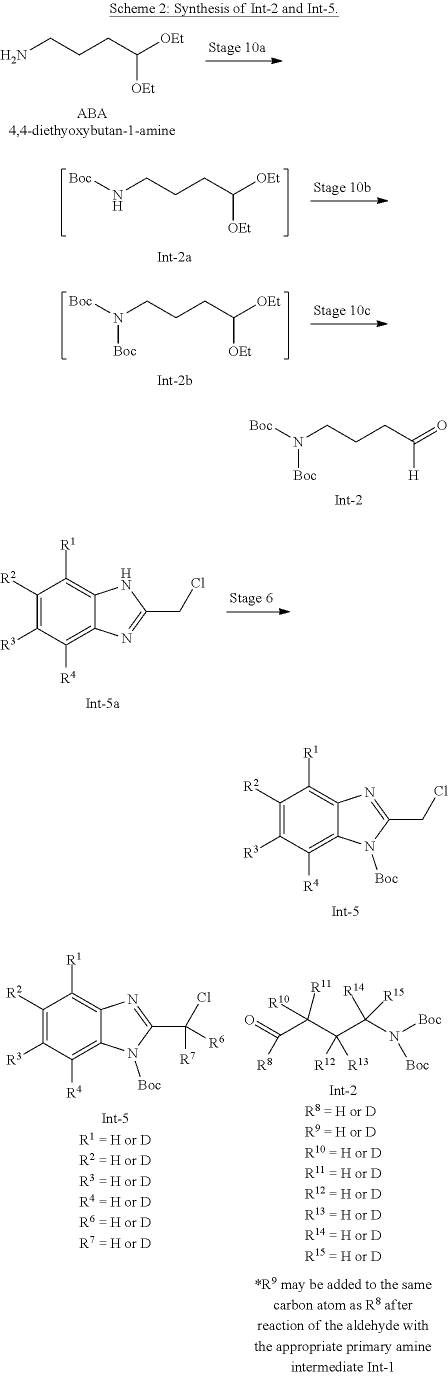

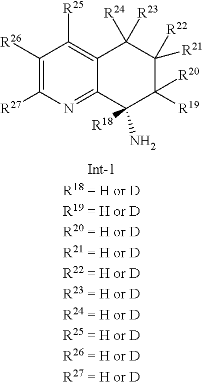

Compound I-4 was prepared and isolated as the TFA salt (compound I-36). These compounds were prepared according to the following scheme:

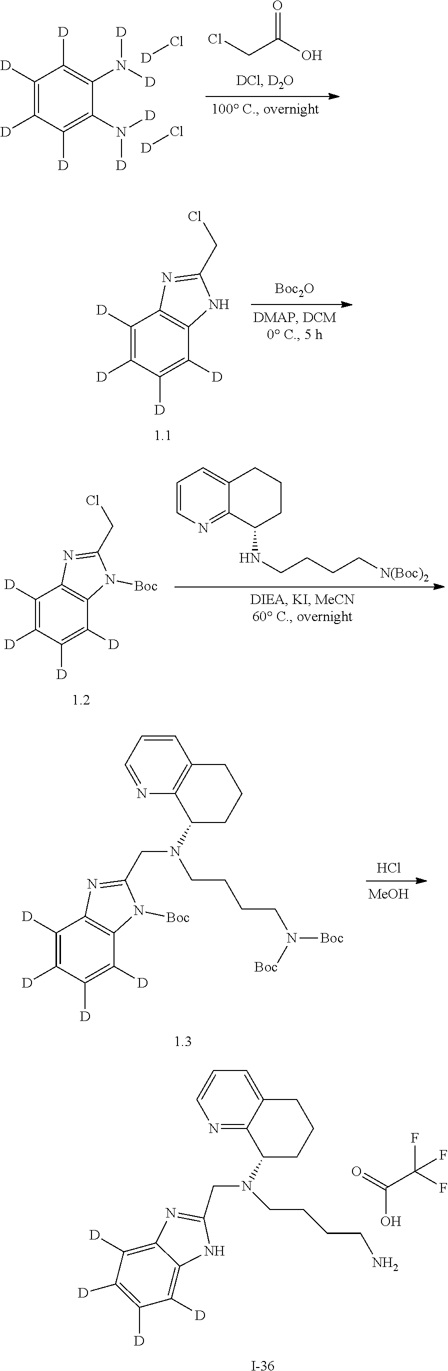

Synthesis of 1.1:

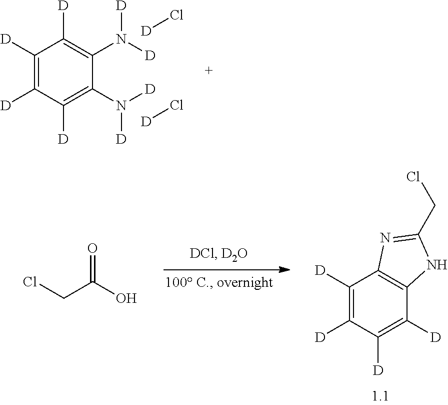

Into a 50-mL 3-necked round-bottom flask was placed 2-chloroacetic acid (2.5 g, 25.13 mmol, 1.00 eq., 95%) and benzene-3,4,5,6-d4-1,2-diamine-N1,N1,N2,N2-d4.DCl salt (3.71 g, 18.44 mmol, 3.00 eq.) in a solution of DCl (5 N in D₂O, 15 mL). The resulting solution was stirred overnight at 100 degrees C. in an oil bath. The resulting mixture was concentrated under vacuum. This resulted in 3.5 g (Crude) of 2-(chloromethyl)-1H-benzo[d]imidazole-4,5,6,7-d4 as purple oil.

Synthesis of 1.2:

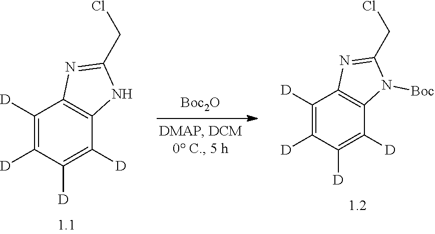 [SMILES: "Boc2O" ; [2H]c1c([2H])c([2H])c2[nH]c(CCl)nc2c1[2H] ; [2H]c1c([2H])c([2H])c2c(nc(CCl)n2C(=O)OC(C)(C)C)c1[2H]]

Into a 50-mL 3-necked round-bottom flask purged and maintained with an inert atmosphere of nitrogen, Boc₂O (13.47 g, 58.63 mmol, 3.00 eq.) was added to a solution of 2-(chloromethyl)-1H-benzo[d]imidazole-4,5,6,7-d4 (3.5 g, 19.49 mmol, 1.00 eq., 95%) in dichloromethane (25 mL) at 0 degrees C. To this solution was added 4-dimethylaminopyridine (251.6 mg, 1.96 mmol, 0.10 eq., 95%). The resulting solution was stirred for 5 h at 0 degrees C. in a water/ice bath. The resulting solution was diluted with 25 mL of DCM. The resulting mixture was washed with 4×25 mL of H₂O. The mixture was dried over anhydrous sodium sulfate. The solids were filtered out. The resulting mixture was concentrated under vacuum. The residue was applied onto a silica gel column with ethyl acetate/petroleum ether (1:1). This resulted in 3.12 g (59%) of tert-butyl 2-(chloromethyl)-1H-benzo[d]imidazole-1-carboxylate-4,5,6,7-d4 as a yellow solid.

Synthesis of 1.3:

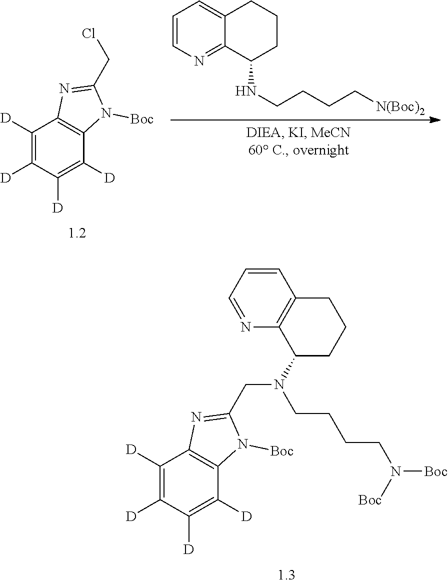

Into a 100-mL 3-necked round-bottom flask purged and maintained with an inert atmosphere of nitrogen, was placed tert-butyl 2-(chloromethyl)-1H-benzo[d]imidazole-1-carboxylate-4,5,6,7-d4 (3.12 g, 10.37 mmol, 1.00 eq., 90%), tert-butyl N-[(tert-butoxy)carbonyl]-N-(4-[[(8S)-5,6,7,8-tetrahydroquinolin-8-yl]amino]butyl)carbamate (9.7 g, 21.96 mmol, 2.00 eq.), KI (210 mg, 1.20 mmol, 0.10 eq., 95%) in MeCN (50 mL) and DIEA (2.236 g, 16.44 mmol, 1.50 eq., 95%). The resulting solution was stirred overnight at 60 degrees C. in an oil bath. The resulting solution was diluted with 20 mL of DCM. The resulting mixture was washed with 4×30 mL of H₂O. The mixture was dried over anhydrous sodium sulfate. The solids were filtered out. The resulting mixture was concentrated under vacuum. This resulted in 8.01 g (crude) of 1.3 as a brown liquid. The crude product was used directly without further purification.

Synthesis of I-36:

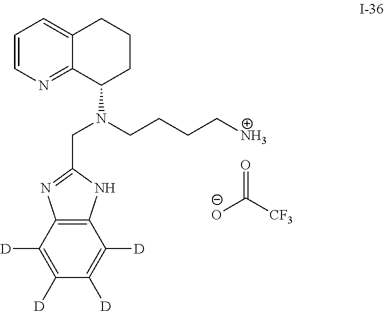 [SMILES: [2H]c1c([2H])c([2H])c2[nH]c(CN(CCCC[NH3+])[C@H]3CCCc4cccnc43)nc2c1[2H] ; O=C([O-])C(F)(F)F]

Into a 100-mL round-bottom flask was placed 1.3 (8.01 g, 11.03 mmol, 1.00 eq. 90%), methanol (30 mL), and a solution of hydrogen chloride in dioxane (1 M, 30 mL). The resulting solution was stirred overnight at 25 degrees C. The resulting mixture was concentrated under vacuum. The residue was dissolved in 8 mL of methanol. The pH value of solution was adjusted to 8 with NH₃ in MeOH. The crude product was purified by Flash-Prep-HPLC with the following conditions (CombiFlash-1): Column, C18; mobile phase, H₂O (0.5% TFA)/CH₃CN=100:0 increasing to H₂O (0.5% TFA)/CH₃CN=85:15 within 15 min; Detector, UV 254 nm. This resulted in 1.4 g (36%) of I-36 as yellow oil. ¹H-NMR (300 MHz, DMSO-d₆, ppm): δ 8.71-8.80 (d, 1H, J=5.4 Hz), 8.23-7.95 (m, 1H), 7.78-7.57 (m, 5H), 4.51-4.44 (m, 3H), 2.85-2.71 (m, 3H), 2.70-2.67 (m, 3H), 2.27-2.24 (m, 1H), 2.14-1.88 (m, 2H), 1.74-1.51 (m, 1H), 1.49-1.39 (m, 4H).

Analytical data for I-36 is shown below.

| Test | Method | Specification | Result |
|---|---|---|---|
| Appearance | Visual inspection | report result | yellow liquid |
| Identification | ¹H NMR | consistent with | consistent with |
| | | structure | structure |
| | LCMS | consistent with | consistent with |
| | | structure | structure |
| Purity | HPLC | >98% | 98.2% (254 nm) |

The ¹⁹F NMR (obtained at 300 MHz in DMSO) showed a single peak at −74.205, consistent with the CF₃CO₂⁻ counterion.

LCMS analyses were run using the following conditions:

Instrument Name: Shimadzu LCMS-2020

Mobile phase A: Water/0.05% TFA

Mobile phase B: Acetonitrile/0.05% TFA

Oven Temperature: 30° C.

Column Name: Waters T3

Length: 100 mm

Internal Diameter: 4.6 mm

Description: 3.0 m

MS (ESI+): Calculated 467.24, Found: 354.30 (M+CF₃CO₂H+1].

Molar mass of I-36: 467.52.

| Event# | Peak# | Ret. Time | m/z | Height | Area |
|---|---|---|---|---|---|
| 1-1 | 1 | 1.222 | 352.00 | 249 | 862 |
| | Total | | | 249 | 862 |
| 1-1 | 1 | 1.218 | 353.00 | 18861 | 57994 |
| | Total | | | 18861 | 57994 |
| 1-1 | 1 | 1.215 | 354.00 | 528014 | 1665057 |
| | Total | | | 528014 | 1665057 |

% Deuterium incorporation was calculated from the mass spectrum data as follows:

$$= [ 1 - \frac{1}{D} \times \frac{\sum {A}_{[ H ]}}{\sum {A}_{[ H ]} + {A}_{[ D ]}} ] \times 100 \\ = \{ 1 - ( 1 / D ) * [ Sum ( {Area}_{352} + {Area}_{353} ) / Sum ( {Area}_{352} + {Area}_{353} + {Area}_{354} ) ] \} * 100 \\ = \{ 1 - ( 1 / 4 ) * [ Sum ( 862 + 57994 ) / Sum ( 862 + 57994 + 1665057 ) ] \} * \\ 100 \% D incorporation in \text{I-36} = 99.15 \%$$

### Where:

A_{[H]}=Peak area of the non-deuterated compound
A_{[D]}=Peak area of deuterated compound
D=The theoretical amount of deuterium

### D_{[0]}=0

Chiral HPLC analysis was also performed as follows:

### Column Name: CHIRALPAK ID-3

### Column Size: 10*0.46 cm, 3.0 m

### Mobile Phase: Hex(0.1% TFA):EtOH=60:40

Flow rate: 1.0 mL/min

### Temperature: 25° C.

The enantiomeric purity was very high. One main peak was detected at RT=1.769 min along with very minor impurities.

### Example 2. Inhibition of Ca²⁺ Signalling in U87.CD4+.CXCR4+ Cells

### Intracellular CXCL-12-Induced Calcium Mobilization Assay

Intracellular calcium mobilization induced by chemokines or chemokine-derived peptides were evaluated using a calcium responsive fluorescent probe and a FLIPR system. The CXCR-4 transfected U87 cell line (U87.CXCR4) cells were seeded in gelatine-coated black-wall 96-well plates at 20,000 cells per well and incubated for 12 hours. Cells were then loaded with the fluorescent calcium probe Fluo-2 acetoxymethyl at 4 μM final concentration in assay buffer (Hanks' balanced salt solution with 20 mM HEPES buffer and 0.2% bovine serum albumin, pH 7.4) for 45 min at 37° C. The intracellular calcium mobilization induced by the CXCL-12 (25-50 ng/mL) was then measured at 37° C. by monitoring the fluorescence as a function of time in all the wells simultaneously using a fluorometric imaging plate reader (FLIPR Tetra, Molecular Devices). The test compounds were added 15 minutes before the addition of CXCL-12 and monitored to see if compounds induced signals by themselves (agonistic properties).

### Chemokine (CXCL12-AF647) Binding Inhibition Assay

Jurkat cells expressing CXCR4 were washed once with assay buffer (Hanks' balanced salt solution with 20 mM HEPES buffer and 0.2% bovine serum albumin, pH 7.4) and then incubated for 15 min at room temperature with the test compounds diluted in assay buffer at dose-dependent concentrations. Subsequently, CXCL12-AF647 (25 ng/mL) was added to the compound-incubated cells. The cells were incubated for 30 min at room temperature. Thereafter, the cells were washed twice in assay buffer, fixed in 1% paraformaldehyde in PBS, and analyzed on the FL4 channel of a FACSCalibur flow cytometer equipped with a 635-nm red diode laser (Becton Dickinson, San Jose, Calif., USA).

The percentages of inhibition of CXCL12-AF647 binding were calculated according to the formula: [1−((MFI−MF_{INC})/(MFI_{PC}−MFI_{NC}))]×100 where MFI is the mean fluorescence intensity of the cells incubated with CXCL12-AF647 in the presence of the inhibitor, MFI_{N}C is the mean fluorescence intensity measured in the negative control (i.e., autofluorescence of unlabeled cells), and MFI_{P}c is the mean fluorescence intensity of the positive control (i.e., cells exposed to CXCL12-AF647 alone).

Results of Assays

TABLE 3
Inhibition of Ca²⁺ Signalling.

| | 1 | 2 | 3 | 4 | AVERAGE |
|---|---|---|---|---|---|
| Compound | IC50 (nM) | IC50 (nM) | IC50 (nM) | IC50 (nM) | IC50 (nM) |
| I-36 | 27.23 | 14.30 | 14.89 | 28.85 | 21.32 |
| X4P-001 (d- | 19.88 | 10.95 | 7.87 | 13.86 | 13.14 |
| mavorixafor) | | | | | |

TABLE 4
Inhibition of CXCL12 Binding in Jurkat CXCR4+ cells.

| | | | AVERAGE |
|---|---|---|---|
| Compound | IC50 (nM) | IC50 (nM) | IC50 (nM) |
| I-36 | 9.5 | 11.1 | 10.3 |
| X4P-001 | 3.2 | 3.0 | 3.1 |
| (d-mavorixafor) | | | |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A compound of formula I: 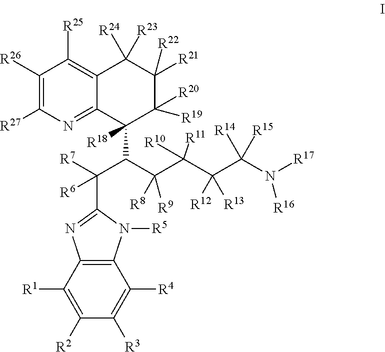
or a pharmaceutically acceptable salt thereof, wherein:
each of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is independently H or D;
provided that at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, and R²⁷ is D.

2. The compound of claim 1, wherein at least one of R¹, R², R³, R⁴, R⁶, R⁷, R⁸, R⁹, R¹⁰, R¹¹, R¹², R¹³, R¹⁴, R¹⁵, R¹⁸, R¹⁹, R²⁰, R²¹, R²², R²³, R²⁴, R²⁵, R²⁶, and R²⁷ is D.

3. The compound of claim 1, wherein R¹ is D.

4. The compound of claim 1, wherein R² is D.

5. The compound of claim 1, wherein R³ is D.

6. The compound of claim 1, wherein R⁴ is D.

7. The compound of claim 1, wherein at least one of R¹, R², R³, and R⁴ has a % deuterium incorporation of about 50% or greater, about 80% or greater, about 90% or greater, or about 95% or greater.

8. The compound of claim 1, wherein the compound is represented by Formula I-a, I-b, I-c, I-d, I-e, I-f, I-g, I-h, or I-i: 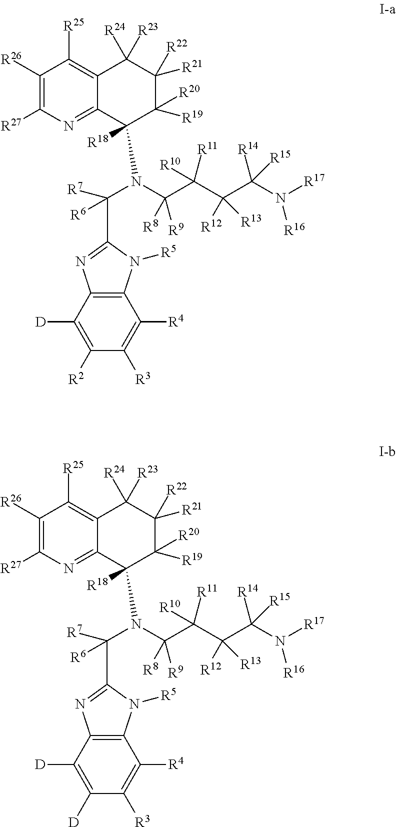 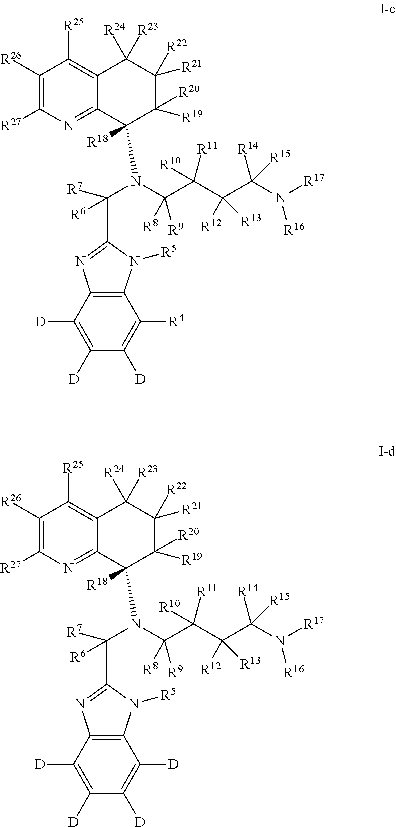 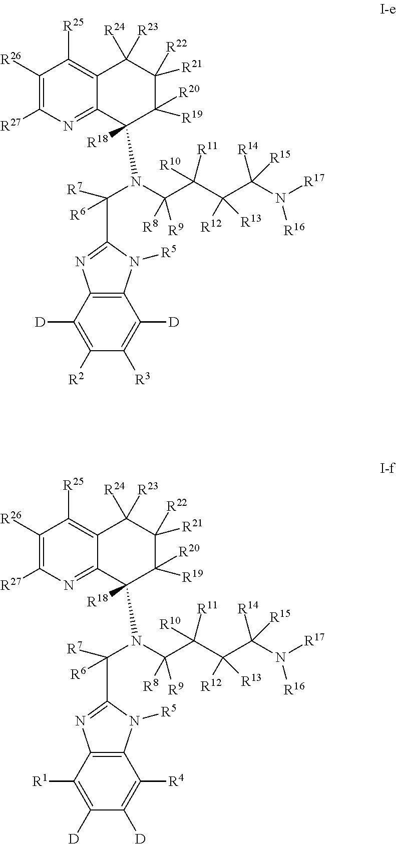 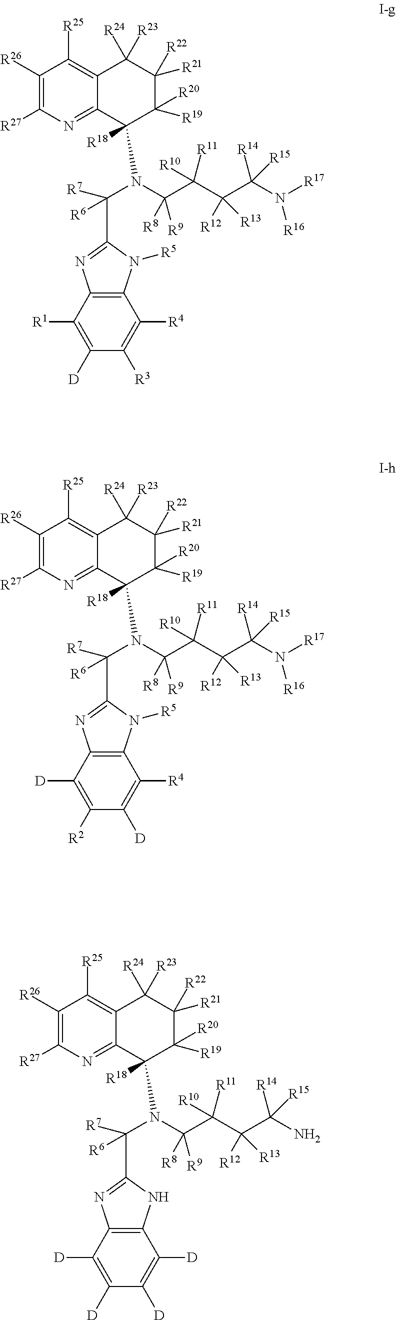
or a pharmaceutically acceptable salt thereof.

9. The compound of claim 8, wherein each position indicated as “D” has a % deuterium incorporation of about 50% or greater, about 80% or greater, about 90% or greater, or about 95% or greater.

10. The compound of claim 1, wherein each of R⁵, R⁶, R⁷, R⁸, R⁹, R¹⁰, R¹¹, R¹², R¹³, R¹⁴, and R¹⁵ is as defined in an entry set forth below:

| Entry | R⁵ | R⁶ | R⁷ | R⁸ | R⁹ | R¹⁰ | R¹¹ | R¹² | R¹³ | R¹⁴ | R¹⁵ |
|---|---|---|---|---|---|---|---|---|---|---|---|
| i | D | H | H | H | H | H | H | H | H | H | H |
| ii | H | D | H | H | H | H | H | H | H | H | H |
| iii | H | H | D | H | H | H | H | H | H | H | H |
| iv | H | H | H | D | H | H | H | H | H | H | H |
| v | H | H | H | H | D | H | H | H | H | H | H |
| vi | H | H | H | H | H | D | H | H | H | H | H |
| vii | H | H | H | H | H | H | D | H | H | H | H |
| viii | H | H | H | H | H | H | H | D | H | H | H |
| ix | H | H | H | H | H | H | H | H | D | H | H |
| x | H | H | H | H | H | H | H | H | H | D | H |
| xi | H | H | H | H | H | H | H | H | H | H | D |
| xii | H | D | H | D | H | H | H | H | H | H | H |
| xiii | H | H | D | H | D | H | H | H | H | H | H |
| xiv | H | H | H | D | H | D | H | H | H | H | H |
| xv | H | H | H | H | D | H | D | H | H | H | H |
| xvi | H | H | H | H | H | D | H | D | H | H | H |
| xvii | H | H | H | H | H | H | D | H | D | H | H |
| xviii | H | H | H | H | H | H | H | D | H | D | H |
| xix | H | H | H | H | H | H | H | H | D | H | D |
| xx | H | D | H | H | D | H | H | H | H | H | H |
| xxi | H | H | D | H | H | D | H | H | H | H | H |
| xxii | H | H | H | D | H | H | D | H | H | H | H |
| xxiii | H | H | H | H | D | H | H | D | H | H | H |
| xxiv | H | H | H | H | H | D | H | H | D | H | H |
| xxv | H | H | H | H | H | H | D | H | H | D | H |
| xxvi | H | H | H | H | H | H | H | D | H | H | D |
| xxvii | D | D | H | H | H | H | H | H | H | H | H |
| xxviii | H | D | D | H | H | H | H | H | H | H | H |
| xxix | H | H | D | D | H | H | H | H | H | H | H |
| xxx | H | H | H | D | D | H | H | H | H | H | H |
| xxxi | H | H | H | H | D | D | H | H | H | H | H |
| xxxii | H | H | H | H | H | D | D | H | H | H | H |
| xxxiii | H | H | H | H | H | H | D | D | H | H | H |
| xxxiv | H | H | H | H | H | H | H | D | D | H | H |
| xxxv | H | H | H | H | H | H | H | H | D | D | H |
| xxxvi | H | H | H | H | H | H | H | H | H | D | D |

or a pharmaceutically acceptable salt thereof.

11. The compound of claim 10, wherein each position indicated as “D” has a % deuterium incorporation of about 50% or greater, about 80% or greater, about 90% or greater, or about 95% or greater.

12. The compound of claim 1, wherein each of R¹⁶, R¹⁷, R⁸, R¹⁹, and R²⁰ is as defined in an entry set forth below:

| Entry | R¹⁶ | R¹⁷ | R¹⁸ | R¹⁹ | R²⁰ |
|---|---|---|---|---|---|
| i | D | H | H | H | H |
| ii | H | D | H | H | H |
| iii | H | H | D | H | H |
| iv | H | H | H | D | H |
| v | H | H | H | H | D |
| vi | D | D | H | H | H |
| vii | H | D | D | H | H |
| viii | H | H | D | D | H |
| ix | H | H | H | D | D |
| x | D | H | D | H | H |
| xi | H | D | H | D | H |
| xii | H | H | D | H | D |
| xiii | D | H | H | D | H |
| xiv | H | D | H | H | D |
| xv | D | H | H | H | D |
| xvi | D | D | D | H | H |
| xvii | H | D | D | D | H |
| xviii | H | H | D | D | D |
| xix | D | H | D | D | H |
| xx | H | D | H | D | D |
| xxi | D | D | D | D | H |
| xxii | H | D | D | D | D |
| xxiii | D | H | D | D | D |
| xxiv | D | D | H | D | D |
| xxv | D | D | D | H | D |
| xxvi | D | D | D | D | D |

or a pharmaceutically acceptable salt thereof.

13. The compound of claim 12, wherein each position indicated as “D” has a % deuterium incorporation of about 50% or greater, about 80% or greater, about 90% or greater, or about 95% or greater.

14. The compound of claim 1, wherein each of R²¹, R²², R²³, R²⁴, R²⁵, R²⁶, and R²⁷ is as defined in an entry set forth below.

| Entry | R²¹ | R²² | R²³ | R²⁴ | R²⁵ | R²⁶ | R²⁷ |
|---|---|---|---|---|---|---|---|
| i | D | H | H | H | H | H | H |
| ii | H | D | H | H | H | H | H |
| iii | H | H | D | H | H | H | H |
| iv | H | H | H | D | H | H | H |
| v | H | H | H | H | D | H | H |
| vi | H | H | H | H | H | D | H |
| vii | H | H | H | H | H | H | D |
| viii | D | D | H | H | H | H | H |
| ix | H | D | D | H | H | H | H |
| x | H | H | D | D | H | H | H |
| xi | H | H | H | D | D | H | H |
| xii | H | H | H | H | D | D | H |
| xiii | H | H | H | H | H | D | D |
| xiv | D | H | D | H | H | H | H |
| xv | H | D | H | D | H | H | H |
| xvi | H | H | D | H | D | H | H |
| xvii | H | H | H | D | H | D | H |
| xviii | H | H | H | H | D | H | D |
| xix | D | H | H | D | H | H | H |
| xx | H | D | H | H | D | H | H |
| xxi | H | H | D | H | H | D | H |
| xxii | H | H | H | D | H | H | D |
| xxiii | D | H | H | H | D | H | H |
| xxiv | H | D | H | H | H | D | H |
| xxv | H | H | D | H | H | H | D |
| xxvi | D | H | H | H | H | D | H |
| xxvii | H | D | H | H | H | H | D |
| xxviii | D | H | H | H | H | H | D |
| xxix | D | D | D | H | H | H | H |
| xxx | H | D | D | D | H | H | H |
| xxxi | H | H | D | D | D | H | H |
| xxxii | H | H | H | D | D | D | H |
| xxxiii | H | H | H | H | D | D | D |

or a pharmaceutically acceptable salt thereof.

15. The compound of claim 14, wherein each position indicated as “D” has a % deuterium incorporation of about 50% or greater, about 80% or greater, about 90% or greater, or about 95% or greater.

16. The compound of claim 1, wherein the compound is selected from any one of the following: 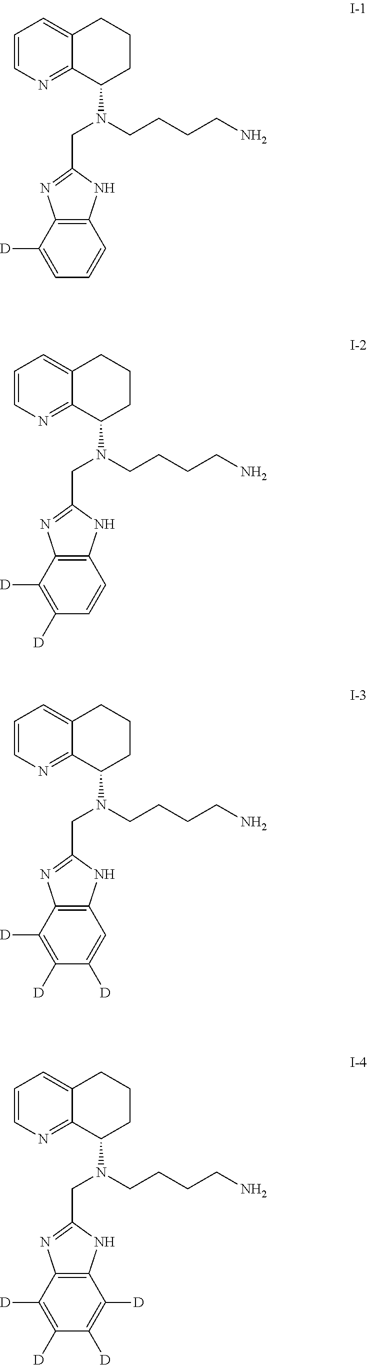 [SMILES: [2H]c1cccc2[nH]c(CN(CCCCN)[C@H]3CCCc4cccnc43)nc12 ; [2H]c1ccc2[nH]c(CN(CCCCN)[C@H]3CCCc4cccnc43)nc2c1[2H] ; [2H]c1cc2[nH]c(CN(CCCCN)[C@H]3CCCc4cccnc43)nc2c([2H])c1[2H] ; [2H]c1c([2H])c([2H])c2[nH]c(CN(CCCCN)[C@H]3CCCc4cccnc43)nc2c1[2H]] 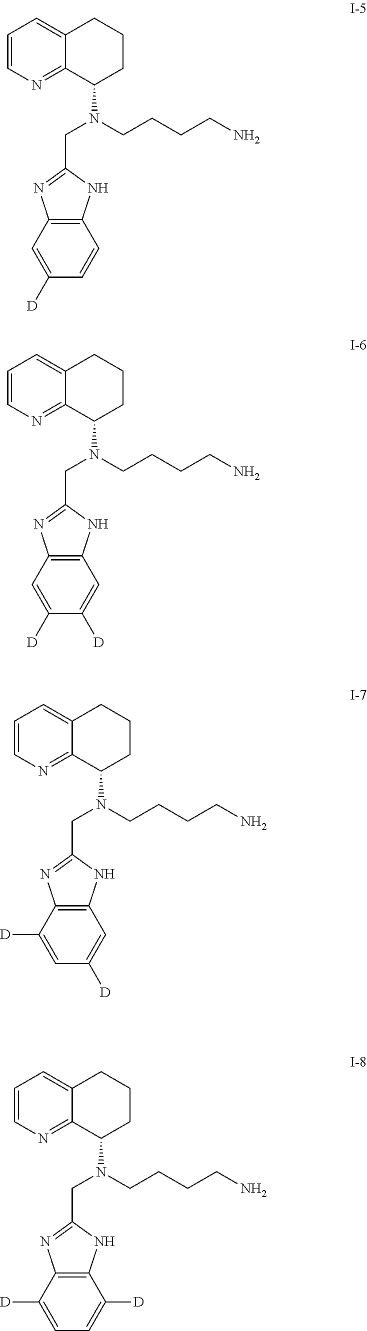 [SMILES: [2H]c1ccc2[nH]c(CN(CCCCN)[C@H]3CCCc4cccnc43)nc2c1 ; [2H]c1cc2nc(CN(CCCCN)[C@H]3CCCc4cccnc43)[nH]c2cc1[2H] ; [2H]c1cc([2H])c2nc(CN(CCCCN)[C@H]3CCCc4cccnc43)[nH]c2c1 ; [2H]c1ccc([2H])c2[nH]c(CN(CCCCN)[C@H]3CCCc4cccnc43)nc12] 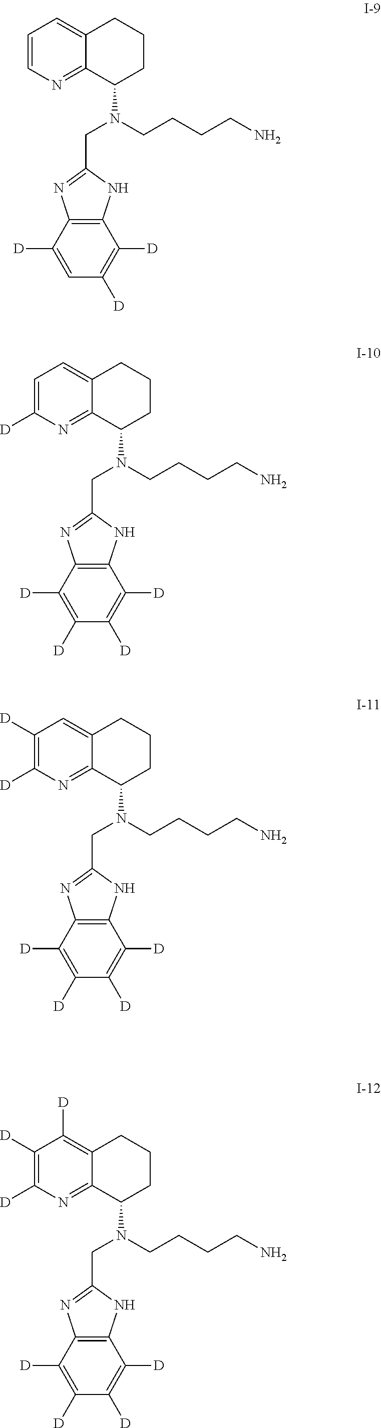 [SMILES: [2H]c1cc([2H])c2nc(CN(CCCCN)[C@H]3CCCc4cccnc43)[nH]c2c1[2H] ; [2H]c1ccc2c(n1)[C@@H](N(CCCCN)Cc1nc3c([2H])c([2H])c([2H])c([2H])c3[nH]1)CCC2 ; [2H]c1cc2c(nc1[2H])[C@@H](N(CCCCN)Cc1nc3c([2H])c([2H])c([2H])c([2H])c3[nH]1)CCC2 ; [2H]c1nc2c(c([2H])c1[2H])CCC[C@@H]2N(CCCCN)Cc1nc2c([2H])c([2H])c([2H])c([2H])c2[nH]1] 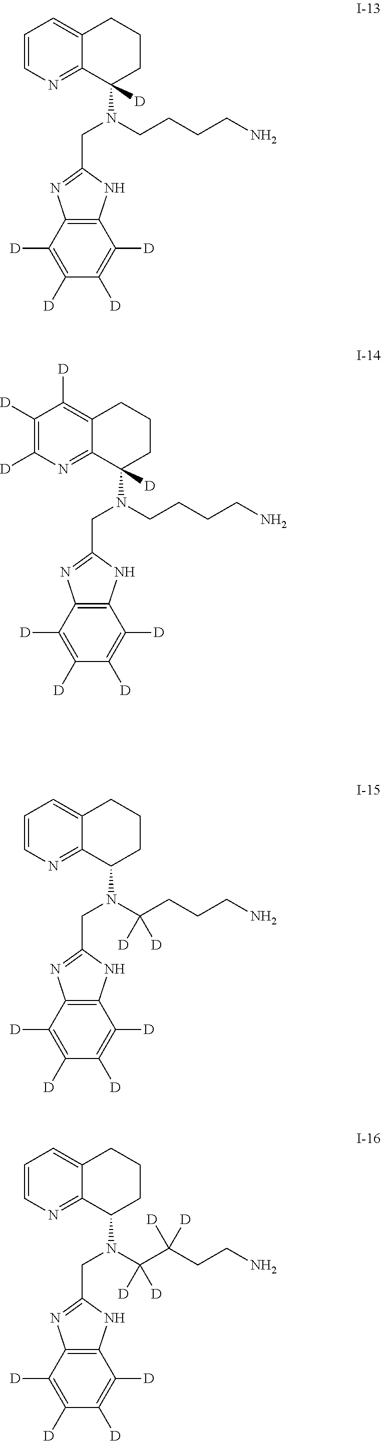 [SMILES: [2H]c1c([2H])c([2H])c2[nH]c(CN(CCCCN)[C@@]3([2H])CCCc4cccnc43)nc2c1[2H] ; [2H]c1nc2c(c([2H])c1[2H])CCC[C@]2([2H])N(CCCCN)Cc1nc2c([2H])c([2H])c([2H])c([2H])c2[nH]1 ; [2H]c1c([2H])c([2H])c2[nH]c(CN([C@H]3CCCc4cccnc43)C([2H])([2H])CCCN)nc2c1[2H] ; [2H]c1c([2H])c([2H])c2[nH]c(CN([C@H]3CCCc4cccnc43)C([2H])([2H])C([2H])([2H])CCN)nc2c1[2H]] 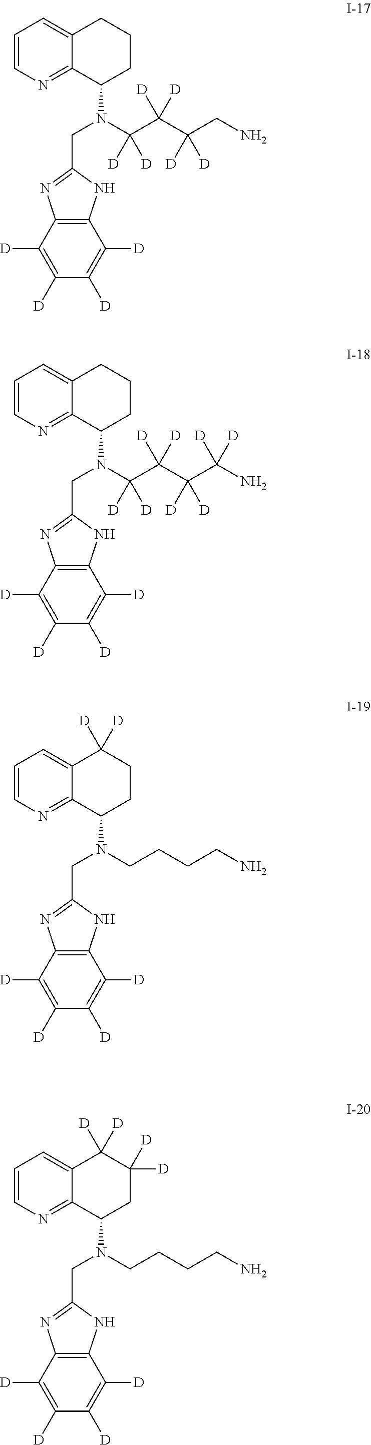 [SMILES: [2H]c1c([2H])c([2H])c2[nH]c(CN([C@H]3CCCc4cccnc43)C([2H])([2H])C([2H])([2H])C([2H])([2H])CN)nc2c1[2H] ; [2H]c1c([2H])c([2H])c2[nH]c(CN([C@H]3CCCc4cccnc43)C([2H])([2H])C([2H])([2H])C([2H])([2H])C([2H])([2H])N)nc2c1[2H] ; [2H]c1c([2H])c([2H])c2[nH]c(CN(CCCCN)[C@H]3CCC([2H])([2H])c4cccnc43)nc2c1[2H] ; [2H]c1c([2H])c([2H])c2[nH]c(CN(CCCCN)[C@H]3CC([2H])([2H])C([2H])([2H])c4cccnc43)nc2c1[2H]] 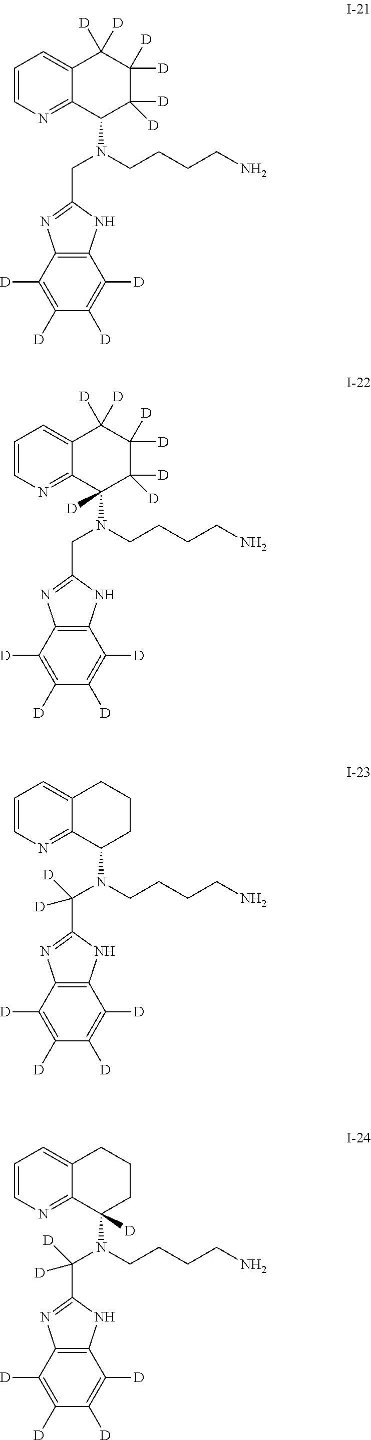 [SMILES: [2H]c1c([2H])c([2H])c2[nH]c(CN(CCCCN)[C@@H]3c4ncccc4C([2H])([2H])C([2H])([2H])C3([2H])[2H])nc2c1[2H] ; [2H]c1c([2H])c([2H])c2[nH]c(CN(CCCCN)[C@]3([2H])c4ncccc4C([2H])([2H])C([2H])([2H])C3([2H])[2H])nc2c1[2H] ; [2H]c1c([2H])c([2H])c2[nH]c(C([2H])([2H])N(CCCCN)[C@H]3CCCc4cccnc43)nc2c1[2H] ; [2H]c1c([2H])c([2H])c2[nH]c(C([2H])([2H])N(CCCCN)[C@@]3([2H])CCCc4cccnc43)nc2c1[2H]] 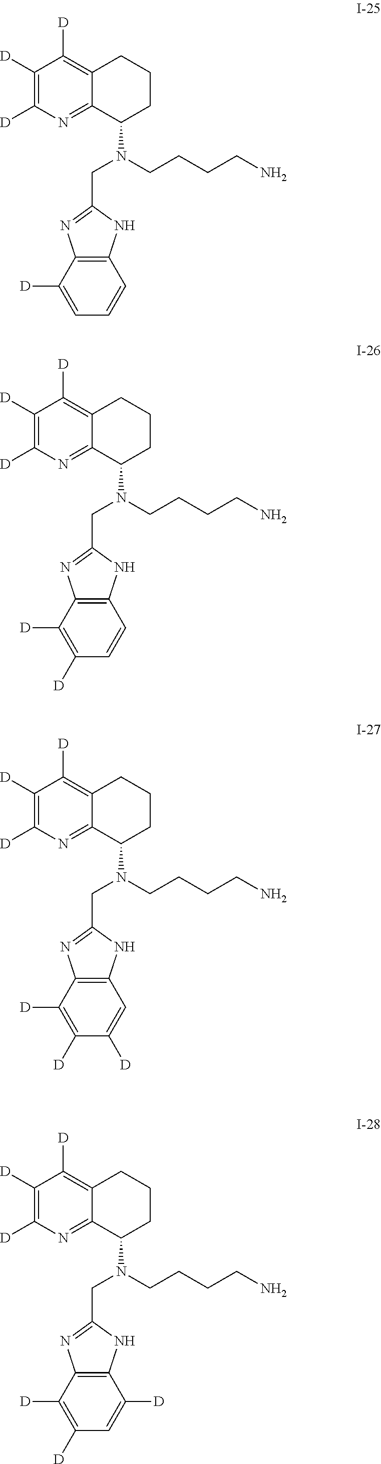 [SMILES: [2H]c1nc2c(c([2H])c1[2H])CCC[C@@H]2N(CCCCN)Cc1nc2c([2H])cccc2[nH]1 ; [2H]c1ccc2[nH]c(CN(CCCCN)[C@H]3CCCc4c3nc([2H])c([2H])c4[2H])nc2c1[2H] ; [2H]c1cc2[nH]c(CN(CCCCN)[C@H]3CCCc4c3nc([2H])c([2H])c4[2H])nc2c([2H])c1[2H] ; [2H]c1cc([2H])c2[nH]c(CN(CCCCN)[C@H]3CCCc4c3nc([2H])c([2H])c4[2H])nc2c1[2H]] 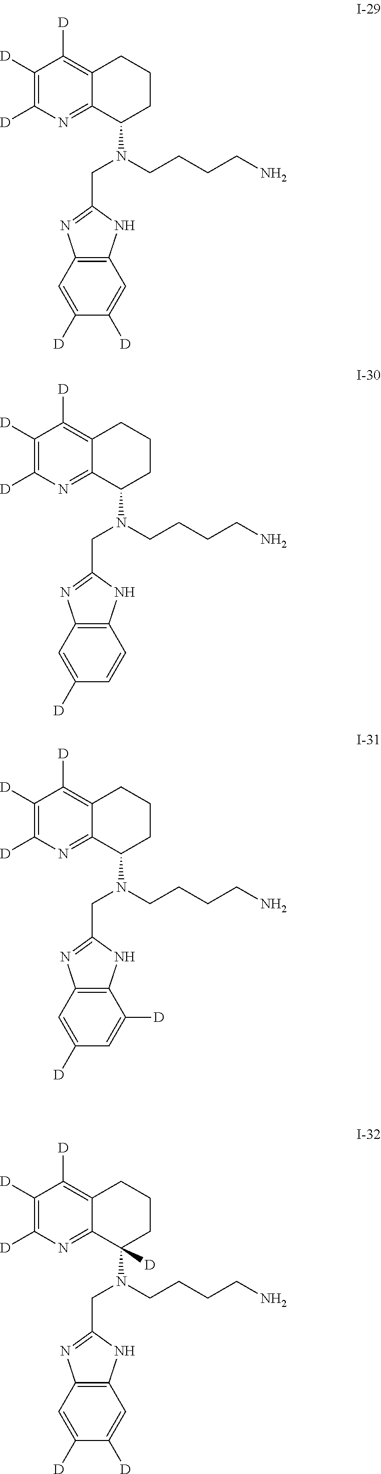 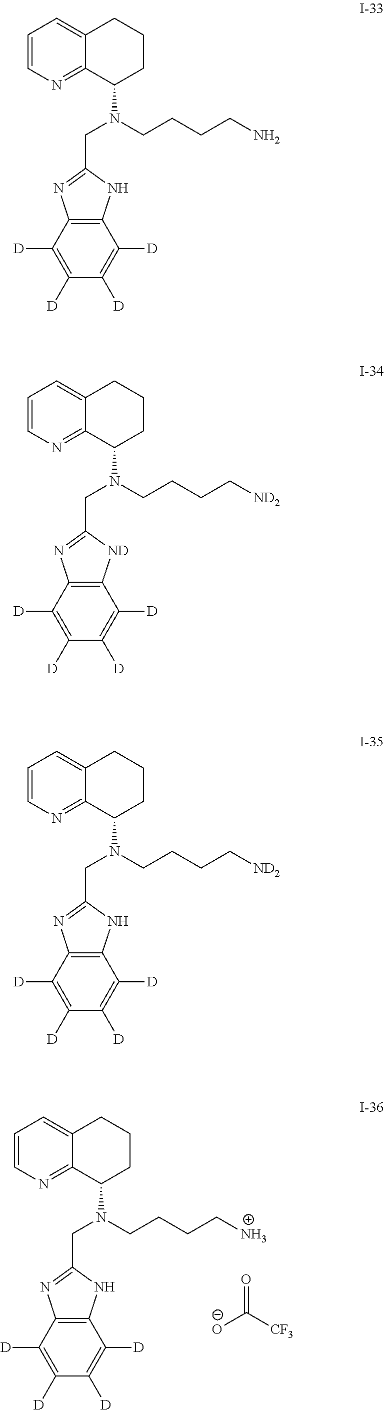
or a pharmaceutically acceptable salt thereof.

17. The compound of claim 16, wherein each position indicated as “D” has a % deuterium incorporation of about 80% or greater, about 90% or greater, or about 95% or greater.

18. A pharmaceutical composition comprising a compound of claim 1, or a pharmaceutically acceptable salt thereof, and a pharmaceutically acceptable excipient, carrier, or vehicle.

19. A method of treating warts, hypogammaglobulinemia, infections, myelokathexis (WHIM) syndrome; severe congenital neutropenia (SCN); G6PC3 deficiency; GATA2 deficiency (Mono MAC syndrome); idiopathic CD4+ T lymphocytopenia (ICL); Wiskott-Aldrich Syndrome, or a cancer, comprising administering to a patient in need thereof an effective amount of a compound of claim 1, or a pharmaceutically acceptable salt thereof.

20. The method of claim 19, wherein the cancer is leukemia selected from acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, and chronic lymphocytic leukemia; polycythemia vera, lymphoma selected from Hodgkin's disease or non-Hodgkin's disease), Waldenstrom's macroglobulinemia, multiple myeloma, heavy chain disease, and solid tumors selected from fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, renal cell cancer, and renal cell carcinoma (RCC).
